(19) United States
(12) Patent Application Publication
(10) Pub. No.: US 2009/0209496 A1
(43) Pub. Date: Aug. 20, 2009

(54) METHODS AND COMPOSITIONS FOR ENHANCING THE EFFICACY OF RTK INHIBITORS

(76) Inventors: David Chaplin, Oxfordshire (GB); Bronwyn G. Siim, Oxford (GB)

Correspondence Address: LAHIVE & COCKFIELD, LLP;FLOOR 30, SUITE 3000, ONE POST OFFICE SQUARE, BOSTON, MA, 02109, US

(21) Appl. No.: 12/372,602

(22) Filed: Feb. 17, 2009

Related U.S. Application Data
(60) Provisional application No. 61/065,898, filed on Feb. 15, 2008.

(51) Int. Cl. A61K 31/661; A61K 31/44; A61K 31/517; A61P 35/00

(52) U.S. Cl. 514/130; 514/350; 514/266.4

(57) ABSTRACT

This invention relates to methods for treating, preventing and/or managing cancer in a subject including enhancing the efficacy of a Receptor Tyrosine Kinase inhibitor (e.g., a small molecule RTK inhibitor, e.g., Sorafenib or Erlotinib) by administering to the subject a Vascular Disrupting Agent (e.g., a Combretastatin or derivative thereof) sequentially or simultaneously in combination with said RTK inhibitor. Pharmaceutical compositions comprising a combination of a RTK inhibitor and a VDA are also provided.

### RELATED APPLICATIONS

This application claims priority to U.S. Provisional Application Ser. No. 61/065,898, entitled “Methods and Compositions for Enhancing the Efficacy of RTK Inhibitors”, filed on Feb. 15, 2008. The entire contents of the aforementioned application are hereby incorporated herein by reference.

### BACKGROUND OF THE INVENTION

The National Cancer Institute has estimated that in the United States alone, 1 in 3 people will be struck with cancer during their lifetime. Moreover approximately 50% to 60% of people contracting cancer will eventually succumb to the disease. The widespread occurrence of this disease underscores the need for improved anticancer regimens for the treatment of malignancy.

Due to the wide variety of cancers presently observed, numerous anticancer agents have been developed to destroy cancer within the body. These compounds are administered to cancer patients with the objective of destroying or otherwise inhibiting the growth of malignant cells while leaving normal, healthy cells undisturbed.

Anticancer agents have been classified based upon their mechanism of action. One class of anticancer agents of recent interest are Receptor Tyrosine Kinase (RTK) Inhibitors. While RTK inhibitors are effective in treating many patients, some patients nevertheless acquire resistance to treatment with RTK inhibitors or relapse following treatment with these agents. Thus, there is an urgent need in the art to provide methods for improving the efficacy of RTK inhibitors.

### SUMMARY OF THE INVENTION

The present invention provides, in part, methods for producing an enhanced antitumor effect wherein a combination of agents is employed. In particular aspects, the methods of the invention comprise the administration (e.g., sequential administration or co-administration) of a Vascular Disrupting Agent (hereinafter, a “VDA”) and a kinase inhibitor (e.g., a Receptor Tyrosine Kinase (RTK) Inhibitor). The methods of the present invention provide advantages such as greater overall therapeutic efficacy of RTK therapy, for example, by preventing tumor regrowth. Further, where a tumor to be treated is not optimally responsive (e.g. resistant) to treatment with either VDA or RTK inhibitor monotherapy, use of the present combination therapy methods can nonetheless provide effective treatment.

In one aspect, the invention provides a method for producing an anti-tumor effect in an patient suffering from a cancer or tumor, the method comprising administering to the patient a VDA and a RTK inhibitor in amounts effective therefor. In one embodiment, a method for inhibiting tumor-associated angiogenesis in a subject treated with a VDA is provided, the method comprising administering to the subject an RTK Inhibitor in amounts effective therefor. In another embodiment, the invention provides a method for destroying tumor vasculature in a subject treated with a RTK inhibitor, the method comprising administering to the subject a VDA in amounts effective therefor. In another embodiment, the invention provides a method for preventing tumor regrowth in a subject suffering from cancer, the method comprising administering to the subject a Vascular Disrupting Agent (VDA) and a RTK inhibitor in amounts effective therefore. In preferred embodiments, the RTK inhibitor is a small molecule RTK inhibitor.

The VDA may be administered at any time relative to administration of said RTK inhibitor. In one embodiment, the VDA and RTK inhibitor may be administered simultaneously to produce a potentiated antitumor effect. In another embodiment the VDA and the RTK inhibitor may be administered sequentially in any order to produce a potentiated antitumor effect. In one preferred embodiment, a RTK inhibitor (e.g. a small molecule RTK inhibitor) is sequentially administered in any order with effective amounts of a VDA (e.g. a combretastatin).

In certain embodiments, the RTK inhibitor is an inhibitor of at least one receptor tyrosine kinase (RTK) selected from the group consisting of: an RTK of the VEGF receptor family, an RTK of the EGF receptor family, an RTK of the RET receptor family, and an RTK of the PDGF receptor family. In exemplary embodiments, the RTK inhibitor is selected from the group consisting of Axitinib, Cediranib, Dasatinib, Erlotinib, Gefitinib, Imatinib, Lapatinib, Lestaurtinib, Nilotinib, Pazopanib, Semaxinib, Sorafenib, Sunitinib, Vatalanib, and Vandetanib. In one preferred embodiment, the RTK inhibitor is Sorafenib. In preferred embodiments, Sorafenib is sequentially administered in any order with an effective amount of a VDA (e.g., a combretastatin). In a still more preferred embodiment, CA4P or CA1P are sequentially or simultaneously administered in any order with an effective of Sorafenib. In another preferred embodiment, the RTK inhibitor is Erlotinib. In preferred embodiments, Erlotinib is sequentially administered in any order with an effective amount of a VDA (e.g., a combretastatin). In a still more preferred embodiment, CA4P or CA1P are sequentially or simultaneously administered in any order with an effective dosage of Erlotinib.

In certain embodiments, the VDA is a combretastatin agent. In one exemplary embodiment, the combretastatin agent is Combretastatin A1 (CA1) or a prodrug or pharmaceutically acceptable salt thereof. In another exemplary embodiment, the combretastatin agent is Combretastatin A4 (CA4) or a prodrug or pharmaceutically acceptable salt thereof. In certain embodiments, the combretastatin agent is a phosphate prodrug. In other embodiments, the combretastatin agent is a compound of Formula II:

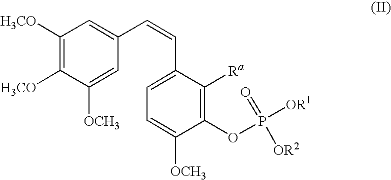

wherein R^{a} is H or OP(O)(OR³)OR⁴; and
   OR¹, OR², OR³ and OR⁴ are each, independently, H, —O⁻QH⁺ or —O⁻M⁺,
wherein M⁺ is a monovalent or divalent metal cation, and Q is, independently:
   a) an amino acid containing at least two nitrogen atoms where one of the nitrogen atoms, together with a proton, forms a quaternary ammonium cation QH⁺; or
   b) an organic amine containing at least one nitrogen atom which, together with a proton, forms a quaternary ammonium cation, QH⁺.

In certain embodiments, Formula II is represented by a compound of Formula III:

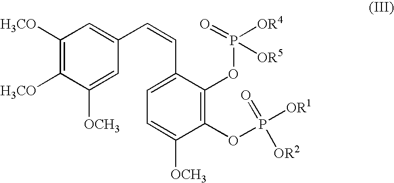

wherein OR¹, OR², OR³ and OR⁴ are each, independently, H, —O⁻QH⁺ or —O⁻M⁺,
wherein M⁺ is a monovalent or divalent metal cation, and Q is, independently:
   a) an amino acid containing at least two nitrogen atoms where one of the nitrogen atoms, together with a proton, forms a quaternary ammonium cation QH⁺; or
   b) an organic amine containing at least one nitrogen atom which, together with a proton, forms a quaternary ammonium cation, QH⁺.

In certain embodiments, wherein the metal cation of Formula III is sodium or potassium. In other embodiment, the metal cation of Formula III is the organic amine is TRIS (ie. tromethamine).

In certain embodiments, the cancer treated with the methods and compositions of the invention is selected from the group consisting of ovarian cancer, fallopian tube cancer, cervical cancer, breast cancer, liver cancer, small cell lung cancer, non-small cell lung cancer, skin cancer, colorectal cancer, esophageal cancer, gastric cancer, leukemia, renal cancer, head and neck cancer, glioma, pancreatic cancer, lymphoma, prostate cancer, and primary cancer of the peritoneum. In certain exemplary embodiments, the cancer is skin cancer or liver cancer.

In certain embodiments, the invention provides a method of treating a subject suffering from cancer, the method comprising administering to the subject a small molecule RTK inhibitor and a combretastatin agent in amounts effective therefor. In one embodiment, the RTK inhibitor and the combretastatin agent are administered simultaneously. In certain exemplary embodiments, the RTK inhibitor is Sorafenib. In other exemplary embodiments, the RTK inhibitor is Erlotinib. In other exemplary embodiments, the combretastatin agent is represented by Formula II.

In certain exemplary embodiments, the invention provides a method of treating a subject suffering from cancer, the method comprising administering to the subject a pharmaceutical composition comprising effective amounts of Sorafenib and CA1P. In other exemplary embodiments, the invention provides a method of treating a subject suffering from cancer, the method comprising thereof by administering to the subject a pharmaceutical composition comprising effective amounts of Sorafenib and CA4P.

In certain exemplary embodiments, the invention provides a method of treating a subject suffering from cancer, the method comprising administering to the subject a pharmaceutical composition comprising effective amounts of Erlotinib and CA1P. In other exemplary embodiments, the invention provides a method of treating a subject suffering from cancer, the method comprising thereof by administering to the subject a pharmaceutical composition comprising effective amounts of Erlotinib and CA4P.

In another aspect, the invention provides a pharmaceutical composition comprising a VDA (e.g., a Combretastatin) and a RTK inhibitor (e.g. a small molecule RTK inhibitor). In one embodiment, the invention provides a pharmaceutical composition for producing an anti-tumor effect in a subject suffering from cancer, comprising effective amounts of a Vascular Disrupting Agent (VDA) and a small molecule RTK inhibitor in a pharmaceutical carrier.

In certain embodiments, the pharmaceutical composition comprises an RTK inhibitor which is an inhibitor of at least one receptor tyrosine kinase (RTK) selected from the group consisting of: an RTK of the VEGF receptor family, an RTK of the EGF receptor family, an RTK of the RET receptor family, and an RTK of the PDGF receptor family.

In one embodiment, the pharmaceutical composition comprises an RTK inhibitor selected from the group consisting of Axitinib, Cediranib, Dasatinib, Erlotinib, Gefitinib, Imatinib, Lapatinib, Lestaurtinib, Nilotinib, Pazopanib, Semaxinib, Sorafenib, Sunitinib, Vatalanib, and Vandetanib; and pharmaceutically acceptable salts thereof. In one preferred embodiment, the RTK inhibitor of the composition is Sorafenib or a pharmaceutically acceptable salt thereof. In another preferred embodiment, the RTK inhibitor of the composition is Erlotinib or a pharmaceutically acceptable salt thereof.

In certain other preferred embodiments, the VDA of the pharmaceutical composition is a combretastatin agent. In one exemplary embodiment, the combretastatin agent of the pharmaceutical composition is Combretastatin A1 (CA1) or a prodrug or pharmaceutically acceptable salt thereof. In another exemplary embodiment, the combretastatin agent of the pharmaceutical composition is Combretastatin A4 (CA4) or a prodrug or pharmaceutically acceptable salt thereof. In other embodiments, the combretastatin agent of the pharmaceutical composition is a phosphate prodrug. In yet other embodiments, the combretastatin agent of the composition is a compound of Formula II:

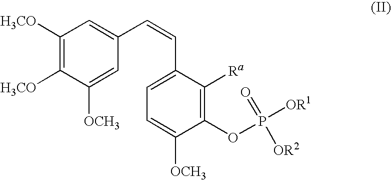

wherein R^{a} is H or OP(O)(OR³)OR⁴; and
   OR¹, OR², OR³ and OR⁴ are each, independently, H, —O⁻QH⁺ or —O⁻M⁺,
wherein M⁺ is a monovalent or divalent metal cation, and Q is, independently:
   a) an amino acid containing at least two nitrogen atoms where one of the nitrogen atoms, together with a proton, forms a quaternary ammonium cation QH⁺; or
   b) an organic amine containing at least one nitrogen atom which, together with a proton, forms a quaternary ammonium cation, QH⁺.

In certain embodiments, the pharmaceutical composition comprises a compound of Formula III:

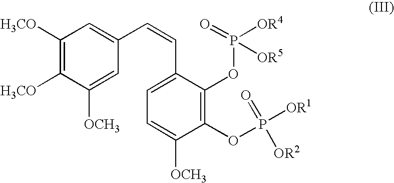

wherein OR¹, OR², OR³ and OR⁴ are each, independently, H, —O⁻QH⁺ or —O⁻M⁺,
wherein M⁺ is a monovalent or divalent metal cation, and Q is, independently:
   a) an amino acid containing at least two nitrogen atoms where one of the nitrogen atoms, together with a proton, forms a quaternary ammonium cation QH⁺; or
   b) an organic amine containing at least one nitrogen atom which, together with a proton, forms a quaternary ammonium cation, QH⁺.

In certain exemplary embodiments, the pharmaceutical composition comprises a combretastatin of Formula III wherein the metal cation is sodium or potassium. In certain exemplary embodiments, the pharmaceutical composition comprises a combretastatin of Formula III wherein the organic amine is TRIS.

In certain particular embodiments, the invention provides a pharmaceutical composition for producing an anti-tumor effect in a subject suffering from cancer, comprising effective amounts of Sorafenib and CA1P in a pharmaceutical carrier. In another particular embodiment, the invention provides a pharmaceutical composition for producing an anti-tumor effect in a subject suffering from cancer, comprising effective amounts of Sorafenib and CA4P in a pharmaceutical carrier.

In other embodiments, the invention provides a pharmaceutical composition for producing an anti-tumor effect in a subject suffering from cancer, comprising effective amounts of Erlotinib and CA1P in a pharmaceutical carrier. In another particular embodiment, the invention provides a pharmaceutical composition for producing an anti-tumor effect in a subject suffering from cancer, comprising effective amounts of Erlotinib and CA4P in a pharmaceutical carrier.

In another aspect, the pharmaceutical composition can be present in a subtherapeutic dose for the individual agent, the agents being more effective when used in combination. Alternatively, each agent can be provided at higher doses for the individual agent, such as those found in the Physician's Desk Reference.

In another aspect, the present invention further provides pharmaceutical kits. Exemplary kits of the invention comprise a first pharmaceutical composition comprising a RTK inhibitor (e.g., a small molecule RTK inhibitor) and a second pharmaceutical composition comprising a VDA (e.g., a Combretastatin) together in a package. The RTK inhibitor and VDA can be present, for example, in a subtherapeutic dose for the individual agent, the agents being effective in combination and providing reduced side effects while maintaining efficacy. Alternatively, each agent can be provided at a higher dose, such as those found for the agent in the Physician's Desk Reference.

In certain aspects, the present invention provides methods of administering a VDA together with a RTK inhibitor in order to potentiate the overall efficacy of the combination. In one embodiment, the VDA and RTK inhibitor are administered simultaneously. In other embodiments, the VDA and RTK inhibitor are administered sequentially. When administered sequentially, a RTK inhibitor can preferably be administered, for example, within 24 hours of the administration of the VDA, such as within 1-24 hours prior, 2-24 hours prior, 3-24 hours prior, 6-24 hours prior, 8-24 hours prior, or 12 to 24 hours prior to administration, or such as within 1-24 hours after, 2-24 hours after, 3-24 hours after, 6-24 hours after, 8-24 hours after, or 12 to 24 hours after administration of the VDA.

### BRIEF DESCRIPTION OF THE DRAWINGS

FIG. 1 depicts the in vivo anti-tumor activity of CA1P (“4503”) alone or in combination with Sorafenib in a mouse model of melanoma.

FIG. 2 depicts the in vivo anti-tumor activity of CA1P (“4503”) alone or in combination with Sorafenib in a mouse model of liver cancer.

### DETAILED DESCRIPTION OF THE INVENTION

The invention is based, on the surprising and unexpected discovery that a new class of chemotherapeutics termed Vascular Disrupting Agents (VDAs) can significantly enhance (e.g., synergistically enhance) the anti-cancer activity of RTK inhibitors. The primary mechanism of action of VDAs (also known as Vascular Targeting Agents (VTAs), Vascular Damaging Agents, or Anti-vascular agents) is “vascular targeting”, in which the neovasculature of tumors is selectively disrupted, resulting in a transient decrease or complete shutdown of tumor blood flow that results in secondary tumor cell death due to hypoxia, acidosis, and/or nutrient deprivation (Dark et al., Cancer Res., 57: 1829-34, (1997); Chaplin et al., Anticancer Res., 19: 189-96, (1999); Hill et al., Anticancer Res., 22(3):1453-8 (2002); Holwell et al., Anticancer Res., 22(2A):707-11, (2002).

So that the invention can be more clearly understood, the following definitions are provided:

### I. DEFINITIONS

As used herein, the term “effective amount” of a compound or pharmaceutical composition refers to an amount sufficient to provide the desired anti-cancer effect or anti-tumor effect in an animal, preferably a human, suffering from cancer. Desired anti-tumor effects include, without limitation, the modulation of tumor growth (e.g. tumor growth delay), tumor size, or metastasis, the reduction of toxicity and side effects associated with a particular anti-cancer agent, the enhancement of tumor necrosis or hypoxia, the reduction of tumor angiogenesis, the reduction of tumor re-growth, reduced tumor retention of CEPs and other pro-angiogenic cells, the amelioration or minimization of the clinical impairment or symptoms of cancer, extending the survival of the subject beyond that which would otherwise be expected in the absence of such treatment, and the prevention of tumor growth in an animal lacking any tumor formation prior to administration, i.e., prophylactic administration.

As used herein, the terms “modulate”, “modulating” or “modulation” refer to changing the rate at which a particular process occurs, inhibiting a particular process, reversing a particular process, and/or preventing the initiation of a particular process. Accordingly, if the particular process is tumor growth or metastasis, the term “modulation” includes, without limitation, decreasing the rate at which tumor growth and/or metastasis occurs; inhibiting tumor growth and/or metastasis, including tumor re-growth following treatment with an anticancer agent; reversing tumor growth and/or metastasis (including tumor shrinkage and/or eradication) and/or preventing tumor growth and/or metastasis.

“Synergistic effect”, as used herein refers to a greater-than-additive anti-cancer effect which is produced by a combination of two drugs, and which exceeds that which would otherwise result from individual administration of either drug alone. One measure of synergy between two drugs is the combination index (CI) method of Chou and Talalay (see Chang et al., Cancer Res. 45: 2434-2439, (1985)) which is based on the median-effect principle. This method calculates the degree of synergy, additivity, or antagonism between two drugs at various levels of cytotoxicity. Where the CI value is less than 1, there is synergy between the two drugs. Where the CI value is 1, there is an additive effect, but no synergistic effect. CI values greater than 1 indicate antagonism. The smaller the CI value, the greater the synergistic effect. Another measurement of synergy is the fractional inhibitory concentration (FIC). This fractional value is determined by expressing the IC₅₀ of a drug acting in combination, as a function of the IC₅₀ of the drug acting alone. For two interacting drugs, the sum of the FIC value for each drug represents the measure of synergistic interaction. Where the FIC is less than 1, there is synergy between the two drugs. An FIC value of 1 indicates an additive effect. The smaller the FIC value, the greater the synergistic interaction.

The term “anticancer agent” as used herein denotes a chemical compound or electromagnetic radiation (especially, X-rays) which is capable of modulating tumor growth or metastasis. When referring to use of such an agent with a combretastatin compound, the term refers to an agent other than a combretastatin compound. Unless otherwise indicated, this term can include one, or more than one, such agents. Thus, the term “anticancer agent” encompasses the use of one or more chemical compounds and/or electromagnetic radiation in the present methods and compositions. Where more than one anticancer agent is employed, the relative time for administration of the combretastatin compound can, as desired, be selected to provide a time-dependent effective tumor concentration of one, or more than one, of the anticancer agents.

As used herein, the term “small molecule” refers to a chemical compound that is less than a few thousand molecular weight. Small molecules do not include macromolecules such as proteins (e.g., antibodies), long chain nucleic acids, or polysaccharides.

As used herein, the term “combretastatin agent” or “combretastatin” denotes at least one member of the combretastatin family of compounds, derivatives or analogs thereof, their prodrugs (preferably phosphate prodrugs) and derivatives thereof, and salts of these compounds. Combretastatins include those anti-cancer compounds isolated from the South African tree Combreturn caffrum, including without limitation, Combretastatins A-1, A-2, A-3, A-4, B-1, B-2, B-3, B-4, D-1, and D-2, and various prodrugs thereof, exemplified by Combretastatin A-4 phosphate (CA4P) compounds, Combretastatin A-1 diphosphate (CA1P) compounds and salts thereof (see for example Pettit et al, Can. J. Chem., (1982); Pettit et al., J. Org. Chem., 1985; Pettit et al., J. Nat. Prod., 1987; Lin et al., Biochemistry, (1989); Pettit et al., J. Med. Chem., 1995; Pettit et al., Anticancer Drug Design, (2000); Pettit et al., Anticancer Drug Design, 16(4-5): 185-93 (2001)). Other exemplary prodrugs of combrestatin agents include the cyclic phosph(oramid)ate prodrugs described in U.S. Pat. Nos. 7,205,404 and 7,303,739, which are incorporated by reference herein. Exemplary combretastatin derivatives retain cis-stilbene as fundamental skeleton and exhibit tubulin polymerization inhibiting activity of 10 micromolar or less (e.g., 1 micromolar, 0.1 micromolar, 10 nanomolar, 1 nanomolar or less).

As used herein, the term combretastatin A-4 phosphate (“CA4P”) denotes as least one of combretastatin A-1 phosphate prodrugs, derivatives thereof, and salts of these compounds. As used herein, the term combretastatin A-1 diphosphate (“CA1P”) compound denotes as least one of combretastatin A-1 diphosphate prodrugs, derivatives thereof, and salts of these compounds.

As used herein, the term “prodrug” refers to a precursor form of the drug which is metabolically converted in vivo to produce the active drug. Thus, for example, combretastatin phosphate prodrug salts administered to an animal in accordance with the present invention undergo metabolic activation and regenerate combretastatin A-4 or combretastatin A-1 in vivo, e.g., following dissociation and exposure to endogenous non-specific phosphatases in the body. A wide variety of methods for the preparation of prodrugs are known to those skilled in the art (see, for example, Pettit and Lippert, Anti-Cancer Drug Design, (2000), 15, 203-216).

A preferred prodrug of the present invention is a phosphate prodrug. As used herein, the term “phosphate prodrug” includes compounds in which a hydroxyl or amino moiety of the active precursor drug is modified with phosphate, phosphoramidate, or amino acid acyl group. The phosphate ester salt moiety may also include (—OP(O)(O-alkyl)₂ or (—OP(O)(O⁻NH₄⁺)₂).

As explained above, the present invention is directed towards a pharmaceutical composition that modulates growth or metastasis of tumors, particularly solid tumors, using a pharmaceutical composition of the present invention, along with methods of modulating tumor growth or metastasis, for example, with a pharmaceutical composition of the present invention.

The term “subject” is intended to include mammals, e.g., humans, dogs, cows, horses, pigs, sheep, goats, cats, mice, rabbits, rats, and transgenic non-human animals. In certain embodiments, the subject is a human, e.g., a human suffering from, at risk of suffering from, or potentially capable of suffering from cancer.

As used herein, the terms “tumor”, “tumor growth” or “tumor tissue” can be used interchangeably, and refer to an abnormal growth of tissue resulting from uncontrolled progressive multiplication of cells and serving no physiological function.

In certain embodiments, the methods and compositions of the invention are used to treat solid tumors. As is well-known in the art, solid tumors are quite distinct from non-solid tumors, such as those found in hemtopoietic-related cancers. A solid tumor can be malignant, e.g. tending to metastasize and being life threatening, or benign. Examples of solid tumors that can be treated or prevented according to a method of the present invention include sarcomas and carcinomas such as, but not limited to: fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, colorectal cancer, gastric cancer, pancreatic cancer, breast cancer, ovarian cancer, fallopian tube cancer, primary carcinoma of the peritoneum, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, liver metastases, bile duct carcinoma, choriocarcinoma, seminoma, embryonal carcinoma, thyroid carcinoma such as anaplastic thyroid cancer, Wilms' tumor, cervical cancer, testicular tumor, lung carcinoma such as small cell lung carcinoma and non-small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, melanoma, neuroblastoma, and retinoblastoma.

In other embodiments, the methods and compositions of the invention are used to treat non-solid tumors. Examples of non-solid tumors include leukemias, such as myeloid leukemias and lymphoid leukemias, myelomas, and lymphomas. Particular forms of non-solid tumors include acute myelitic leukemia (AML), acute lymphatic leukemia (ALL), multiple myeloma (MM), chronic myelogenous leukemia (CML), hairy cell leukemia (HCL), acute promyelocytic leukemia (APL), and chronic lymphocytic leukemia (CLL).

In other embodiments, tumors comprising dysproliferative changes (such as metaplasias and dysplasias) can be treated or prevented with a pharmaceutical composition or method of the present invention in epithelial tissues such as those in the cervix, esophagus, and lung. Thus, the present invention provides for treatment of conditions known or suspected of preceding progression to neoplasia or cancer, in particular, where non-neoplastic cell growth consisting of hyperplasia, metaplasia, or most particularly, dysplasia has occurred (for review of such abnormal growth conditions, see Robbins and Angell, 1976, Basic Pathology, 2d Ed., W.B. Saunders Co., Philadelphia, pp. 68 to 79). Hyperplasia is a form of controlled cell proliferation involving an increase in cell number in a tissue or organ, without significant alteration in structure or function. For example, endometrial hyperplasia often precedes endometrial cancer. Metaplasia is a form of controlled cell growth in which one type of adult or fully differentiated cell substitutes for another type of adult cell. Metaplasia can occur in epithelial or connective tissue cells. Atypical metaplasia involves a somewhat disorderly metaplastic epithelium. Dysplasia is frequently a forerunner of cancer, and is found mainly in the epithelia; it is the most disorderly form of non-neoplastic cell growth, involving a loss in individual cell uniformity and in the architectural orientation of cells. Dysplastic cells often have abnormally large, deeply stained nuclei, and exhibit pleomorphism. Dysplasia characteristically occurs where there exists chronic irritation or inflammation, and is often found in the cervix, respiratory passages, oral cavity, and gall bladder. For a review of such disorders, see Fishman et al., 1985, Medicine, 2d Ed., J. B. Lippincott Co., Philadelphia.

Other examples of tumors that are benign and can be treated or prevented in accordance with a method of the present invention include arteriovenous (AV) malformations, particularly in intracranial sites and myoleomas.

As used herein, the term “pharmaceutically acceptable salt” includes salts that are physiologically tolerated by a subject. Such salts are typically prepared from an inorganic and/or organic acid. Examples of suitable inorganic acids include, but are not limited to, hydrochloric, hydrobromic, hydroiodic, nitric, sulfuric, and phosphoric acid. Organic acids may be aliphatic, aromatic, carboxylic, and/or sulfonic acids. Suitable organic acids include, but are not limited to, formic, acetic, propionic, succinic, camphorsulfonic, citric, fumaric, gluconic, lactic, malic, mucic, tartaric, para-toluenesulfonic, glycolic, glucuronic, maleic, furoic, glutamic, benzoic, anthranilic, salicylic, phenylacetic, mandelic, pamoic, methanesulfonic, ethanesulfonic, pantothenic, benzenesulfonic (besylate), stearic, sulfanilic, alginic, galacturonic, and the like. Other pharmaceutically acceptable salts include alkali metal cations such as Na, K, Li; alkali earth metal salts such as Mg or Ca; or organic amine salts such as those disclosed in PCT International Application Nos. WO02/22626 or WO00/48606 and U.S. Pat. Nos. 6,855,702 and 6,670,344, which are incorporated herein by reference in their entireties. Particularly preferred salts include organic amine salts such tromethamine (TRIS) and amino acid salts such as histidine. Other exemplary salts which can be synthesized using the methods of the invention include those described in U.S. Pat. No. 7,018,987, which is incorporated by reference herein.

### II. VASCULAR DISRUPTING AGENTS (VDAS)

Vascular Disrupting Agents (“VDAs”), also known as vascular disrupting agents or vascular targeting agents, are a separate class of antivascular chemotherapeutics. In contrast to anti-angiogenic drugs which disrupt the new microvessel formation of developing tumors, VDAs attack solid tumors by selectively targeting the established tumor vasculature and causing extensive shutdown of tumor blood flow. A single dose of a VDA can cause a rapid and selective shutdown of the tumor neovasculature within a period of minutes to hours, leading eventually to tumor necrosis by induction of hypoxia and nutrient depletion. This vascular-mediated cytotoxic mechanism of VDA action is quite divorced from that of anti-angiogenic agents, which inhibit the formation of new tumor vascularization rather than interfering with the existing tumor vasculature. Other agents have been known to disrupt tumor vasculature, but differ in that they also manifest substantial normal tissue toxicity at their maximum tolerated dose. In contrast, genuine VDAs retain their vascular shutdown activity at a fraction of their maximum tolerated dose. It is thought that tubulin-binding VDAs selectively destabilize the microtubule cytoskeleton of tumor endothelial cells, causing a profound alteration in the shape of the cell which ultimately leads to occlusion of the tumor blood vessel and shutdown of blood flow to the tumor (Kanthou et al., Blood, 2002; Cooney et al., Curr Oncol Rep. 2005 7(2):90-5; Chaplin et al., Curr Opin Investig Drugs, (2006), 7(6):522-8).

A particularly promising subclass of VDAs are Combretastatins. Derived from the South African tree Combreturn caffrum, combretastatins such as Combretastatin A-4 (CA-4) were initially identified in the 1980's as a potent inhibitors of tubulin polymerization. CA-4, and other combretastatins (e.g. CA-1) have been shown to bind a site at or near the colchicine binding site on tubulin with high affinity. In vitro studies clearly demonstrated that combretastatins are potent cytotoxic agents against a diverse spectrum of tumor cell types in culture. CA4P and CA1P, respective phosphate prodrugs of CA-4 and CA-1, were subsequently developed to combat problems with aqueous insolubility (see U.S. Pat. Nos. 4,996,237; 5,409,953; and 5,569,786, each of which is incorporated herein by reference). Surprisingly, CA1P and CA4P have also been shown to cause a rapid and acute shutdown of the blood flow to tumor tissue that is separate and distinct from the anti-proliferative effects of the agents on tumor cells themselves. A number of studies have shown that combretastatins cause extensive shut-down of blood flow within the tumor microvasculature, leading to secondary tumor cell death (Dark et al., Cancer Res., 57: 1829-34, (1997); Chaplin et al., Anticancer Res., 19: 189-96, (1999); Hill et al., Anticancer Res., 22(3):1453-8 (2002); Holwell et al., Anticancer Res., 22(2A):707-11, (2002). Blood flow to normal tissues is generally far less affected by CA4P and CA1P than blood flow to tumors, although blood flow to some organs, such as spleen, skin, skeletal muscle and brain, can be inhibited (Tozer et al., Cancer Res., 59: 1626-34 (1999)).

In light of the novel, non-cytotoxic, mode of action of combretastatins, there is considerable interest in exploiting the novel “vascular targeting” of these agents for cancer treatment. Recently, single agent efficacy was reported for CA4P using a frequent dosing regimen. Another report suggested that large tumors can, in some cases, be more responsive to CA4P therapy than small tumors. However, many tumors harvested from animals treated with CA4P reveal central necrosis surrounded by a rim of viable cells (Dark et al., Cancer Res., 57: 1829-34, (1997); Chaplin et al., Anticancer Res., 19: 189-96, (1999)). This rim of surviving cells is most likely a consequence of the shared normal vessel circulation between the perimeter of tumours and neighbouring normal tissue.

Exemplary combretastatin salts contemplated for use in the methods of the invention are described in WO 99/35150; WO 01/81355; U.S. Pat. Nos. 6,670,344; 6,538,038; 5,569,786; 5,561,122; 5,409,953; 4,996,237 which are incorporated herein by reference in their entirety.

Exemplary combretastatin derivatives or analogs of combretastatins are described in Singh et al., J. Org. Chem., 1989; Cushman et al, J. Med. Chem., 1991; Getahun et al, J. Med. Chem., 1992; Andres et al, Bioorg. Med. Chem. Lett., 1993; Mannila, et al., Liebigs. Ann. Chem., 1993; Shirai et al., Bioorg. Med. Chem. Lett., 1994; Medarde et al., Bioorg. Med. Chem. Lett., 1995; Wood et al, Br. J. Cancer, 1995; Bedford et al., Bioorg. Med. Chem. Lett., 1996; Dorr et al., Invest. New Drugs, 1996; Jonnalagadda et al., Bioorg. Med. Chem. Lett., 1996; Shirai et al., Heterocycles, 1997; Aleksandrzak, et al., Anticancer Drugs, 1998; Chen et al., Biochem. Pharmacol., 1998; Ducki et al., Bioorg. Med. Chem. Lett., 1998; Hatanaka et al., Bioorg. Med. Chem. Lett., 1998; Medarde et al., Eur. J. Med. Chem., 1998; Medina et al., Bioorg. Med. Chem. Lett., 1998; Ohsumi et al., Bioorg. Med. Chem. Lett., 1998; Ohsumi et al., J. Med. Chem., 1998; Pettit, et al., J. Med. Chem., 1998; Shirai et al., Bioorg. Med. Chem. Lett., 1998; Banwell et al., Aust. J. Chem., 1999; Medarde et al., Bioorg. Med. Chem. Lett., 1999; Shan et al., PNAS, 1999; Combeau et al., Mol. Pharmacol., 2000; Pettit et al., J. Med. Chem., 2000; Pinney et al., Bioorg. Med. Chem. Lett., 2000; Flynn et al., Bioorg. Med. Chem. Lett., 2001; Gwaltney et al., Bioorg. Med. Chem. Lett., 2001; Lawrence et al., 2001; Nguyen-Hai et al., Bioorg. Med. Chem. Lett., 2001; Xia et al., J. Med. Chem., 2001; Tahir et al., Cancer Res., 2001; Wu-Wong et al., Cancer Res., 2001; Janik et al, Biooorg. Med. Chem. Lett., 2002; Kim et al., Bioorg Med Chem Lett., 2002; Li et al., Biooorg. Med. Chem. Lett., 2002; Nam et al., Bioorg. Med. Chem. Lett., 2002; Wang et al., J. Med. Chem. 2002; Hsieh et al., Biooorg. Med. Chem. Lett., 2003; Hadimani et al., Bioorg. Med. Chem. Lett., 2003; Mu et al., J. Med. Chem, 2003; Nam et al., Curr. Med. Chem., 2003; Pettit et al, J. Med. Chem., 2003; Gaukroger et al., Org Biomol Chem. 2003; Bailly et al., J Med Chem. 2003; Sun et al., Anticancer Res. 2004; Sun et al., Bioorg Med Chem Lett. 2004; Liou et al., J Med Chem. 2004; Perez-Melero et al., Bioorg Med Chem Lett. 2004; Liou et al., J Med Chem. 2004; Mamane et al., Chemistry. 2004; De Martini et al, J Med Chem. 2004; Ducki et al, J Med Chem. 2005; Maya et al., J Med Chem. 2005; Medarde et al., J Enzyme Inhib Med Chem. 2004; Simoni et al, J Med Chem. 2005; Sanchez et al., Bioorg Med Chem. 2005; Vongvanich et al., Planta Med. 2005; Tron et al., J Med Chem. 2005; Borrel et al., Bioorg Med Chem. 2005; Hsieh et al., Curr Pharm Des. 2005; Lawrence et al, Curr Pharm Des. 2005; Hadfield et al., Eur. J. Med. Chem. 2005; Coggioloa et al., Bioorg Med Chem Lett. 2005; Kaffy et al., Org Biomol Chem. 2005; Mateo et al, J Org Chem. 2005; LeBlanc et al., Bioorg Med Chem. 2005; Srivistava et al., Bioorg Med Chem. 2005; Nguyen et al., J Med Chem. 2005; Kong et al., Chem Biol. 2005; Li et al, Bioorg Med Chem Lett. 2005; Pettit et al, J Nat Prod. 2005; Nicholson et al, Anticancer Drugs. 2006; Monk et al., Bioorg Med Chem. 2006; De Martino et al., J Med Chem. 2006; Peifer et al., J Med Chem. 2006; Kaffy et al., Bioorg Med Chem. 2006; Banwell et al., Bioorg Med Chem. 2006; Dupeyre et al., Bioorg Med Chem. 2006 Simoni et al, J Med Chem. 2006; Tron et al., J Med Chem. 2006; Romagnoli et al, J Med Chem. 2006; Pandit et al., Bioorg Med Chem. 2006; Nakamura et al., Chem Med Chem. 2006; Pirali et al., J Med Chem. 2006; Bellina et al, Bioorg Med Chem Lett. 2006; Hu et al, J Med Chem. 2006; Chang et al., J Med Chem. 2006; Thomson et al., Mol Cancer Ther. 2006; Fortin et al., Bioorg Med Chem Lett., 2007; Duan et al., J Med Chem., 2007; Zhang et al., J Med Chem. 2007; Wu et al., Bioorg Med Chem Lett. 2007; Sun et al., Bioorg Med Chem Lett. 2007, WO 07/140,662; WO 07/059,118; WO 06/138427; WO 06/036743; WO 05/007635, WO 03/040077, WO 03/035008, WO 02/50007, WO 02/14329; WO 01/12579, WO 01/09103, WO 01/81288, WO 01/84929, WO 00/48590, WO 00/73264, WO 00/06556, WO 00/35865, WO 99/34788, WO 99/48495, WO 92/16486, U.S. Pat. Nos. 7,312,241; 7,223,747; 7,220,784; 7,135,502; 7,125,906; 7,105,695; 7,105,501; 7,087,627; 7,030,123; 7,078,552; 7,030,123; 7,018,987; 6,992,106; 6,919,324; 6,846,192, 6,855,702; 6,849,656; 6,794,384; 6,787,672, 6,777,578, 6,723,858, 6,720,323, 6,433,012, 6,423,753, 6,201,001, 6,150,407, 6,169,104, 5,731,353, 5,674,906, 5,430,062, 5,525,632, 4,996,237 and 4,940,726, each of which are incorporated herein by reference in their entirety.

In one exemplary embodiment, a combretastatin derivate is the amine or serinamide derivative of CA4, e.g. AVE8032 (Aventis Pharma, France). In another exemplary embodiment, a combretastatin derivative is ZD6126 (AstraZeneca, UK).

In particular embodiments, a combretastatin derivative is a compound of Formula I:

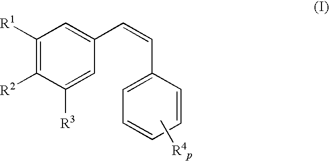

wherein
each of R¹, R² and R³, independently of the others, is selected from the group consisting of hydrogen, C₁₋₆ alkoxy, and halogen, wherein at least two of R¹, R² and R³ are non-hydrogen;
R⁴ is selected from the group consisting of R⁵, R⁶, R⁵ substituted with one or more of the same or different R⁷ or R⁶, —OR⁷ substituted with one or more of the same or R⁷ or R⁶, —B(OR⁷)₂, —B(NR⁸R⁸)₂, —(CH₂)ₘ—R⁶, —(CHR⁷)ₘ—R⁶, —O—(CH₂)ₘ—R⁶, —S—(CH₂)ₘ—R⁶, —O—CHR⁷R⁶, —O—CR⁷(R⁶)₂, —O—(CHR⁷)ₘ—R⁶, —O—(CH₂)ₘ—CH[(CH₂)ₘR⁶]R⁶, —S—(CHR⁷)ₘ—R⁶, —C(O)NH—(CH₂)ₘ—R⁶, —C(O)NH—(CHR⁷)ₘ—R⁶, —O—(CH₂)ₘ—C(O)NH—(CH₂)ₘ—R⁶, —S—(CH₂)ₘ—C(O)NH—(CH₂)ₘ—R⁶, —O—(CHR⁷)ₘ—C(O)NH—(CHR⁷)ₘ—R⁶, —S—(CHR⁷)ₘ—C(O)NH—(CHR⁷)ₘ—R⁶, —NH—(CH₂)ₘ—R⁶, —NH—(CHR⁷)ₘ—R⁶, —NH[(CH₂)ₘR⁶], —N[(CH₂)ₘR⁶]₂, —NH—C(O)—NH—(CH₂)ₘ—R⁶, —NH—C(O)—(CH₂)ₘ—CHR⁶R⁶ and —NH—(CH₂)ₘ—C(O)—NH—(CH₂)ₘ—R⁶;
each R⁵ is independently selected from the group consisting of C₁₋₆ alkyl, C₃₋₈ cycloalkyl, C₄₋₁₁ cycloalkylalkyl, C₅₋₁₀ aryl, C₆₋₁₆ arylalkyl, 2-6 membered heteroalkyl, 3-8 membered cycloheteroalkyl, 4-11 membered cycloheteroalkylalkyl, 5-10 membered heteroaryl, 6-16 membered heteroarylalkyl, phosphate, phosphate ester, phosphonate, phosphorodiamidate, phosphoramidate monoester, phosphoramidate diester, cyclic phosphoramidate, cyclic phosphorodiamidate, and phosphonamidate
each R⁶ is a suitable group independently selected from the group consisting of ═O, —OR⁷, C₁₋₃ haloalkyloxy, —OCF₃, ═S, —SR⁷, ═NR⁷, ═NOR⁷, —NR⁸R⁸, halogen, —CF₃, —CN, —NC, —OCN, —SCN, —NO, —NO₂, ═N₂, —N₃, —S(O)R⁷, —S(O)₂R⁷, —S(O)₂OR⁷, —S(O)NR⁸R⁸, —S(O)₂NR⁸R⁸, —OS(O)R⁷, —OS(O)₂R⁷, —OS(O)₂OR⁷, —OS(O)₂NR⁸R⁸, —C(O)R⁷, —C(O)OR⁷, —C(O)NR⁸R⁸, —C(NH)NR⁸R⁸, —C(NR⁷)NR⁸R⁸, —C(NOH)R⁷, —C(NOH)NR⁸R⁸, —OC(O)R⁷, —OC(O)OR⁷, —OC(O)NR⁸R⁸, —OC(NH)NR⁸R⁸, —OC(NR⁷)NR⁸R⁸, —[NHC(O)]ₙR⁷, —[NR⁷C(O)]ₙR⁷, —[NHC(O)]ₙOR⁷, —[NR⁷C(O)]ₙOR⁷, —[NHC(O)]ₙNR⁸R⁸, —[NR⁷C(O)]ₙNR⁸R⁸, —[NHC(NH)]ₙNR⁸R⁸ and —[NR⁷C(NR⁷)]ₙNR⁸R⁸;
each R⁷ is independently selected from the group consisting of hydrogen, C₁₋₆ alkyl, C₃₋₈ cycloalkyl, C₄₋₁₁ cycloalkylalkyl, C₅₋₁₀ aryl, C₆₋₁₆ arylalkyl, 2-6 membered heteroalkyl, 3-8 membered cycloheteroalkyl, 4-11 membered cycloheteroalkylalkyl, 5-10 membered heteroaryl, 6-16 membered heteroarylalkyl, phosphate, phosphate ester, phosphonate, phosphorodiamidate, phosphoramidate monoester, phosphoramidate diester, cyclic phosphoramidate, cyclic phosphorodiamidate, and phosphonamidate;
each R⁸ is independently R⁷ or, alternatively, two R⁸ are taken together with the nitrogen atom to which they are bonded to form a 5 to 8-membered cycloheteroalkyl or heteroaryl which may optionally include one or more of the same or different additional heteroatoms and which may optionally be substituted with one or more of the same or different R⁷ or suitable R⁶ groups;
each m independently is an integer from 1 to 3;
each n independently is an integer from 0 to 3;
p is an integer from 1 to 5, and

wherein two adjacent R⁴ groups and their intervening atoms are bonded to form a 5-8 membered ring fused to the central phenyl group.

In a particular embodiment, the combrestatin agent is a phosphate prodrug of a combretastatin agent. An exemplary phosphate prodrug is a compound of the Formula II:

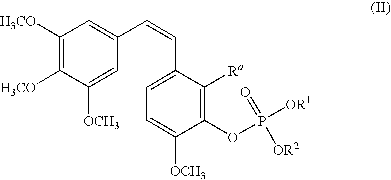

wherein

R^{a} is H or OP(O)(OR³)OR⁴; and

OR¹, OR², OR³ and OR⁴ are each, independently, H, —O⁻QH⁺ or —O⁻M⁺, wherein M⁺ is a monovalent or divalent metal cation, and Q is, independently:

a) an amino acid containing at least two nitrogen atoms where one of the nitrogen atoms, together with a proton, forms a quaternary ammonium cation QH⁺; or

b) an organic amine containing at least one nitrogen atom which, together with a proton, forms a quaternary ammonium cation, QH⁺.

In one embodiment of Formula I, R^{a} is H, one of OR¹ and OR² is hydroxyl, and the other is —O⁻QH⁺ where Q is L-histidine. In another embodiment of Formula II, R^{a} is H, one of OR¹ and OR² is hydroxyl and the other is —O⁻QH⁺ and Q is tris(hydroxymethyl)amino methane (“TRIS”).

In another embodiment of Formula II, R^{a} is H or OP(O)(OR³)OR⁴, and R¹, R², R³ and R⁴ are each, independently, an aliphatic organic amine, alkali metals, transition metals, heteroarylene, heterocyclyl, nucleoside, nucleotide, alkaloid, amino sugar, amino nitrile, or nitrogenous antibiotic.

In another embodiment of Formula II, R¹, R², R³ and R⁴ are each, independently, Na, TRIS, histidine, ethanolamine, diethanolamine, ethylenediamine, diethylamine, triethanolamine, glucamine, N-methylglucamine, ethylenediamine, 2-(4-imidazolyl)-ethylamine, choline, or hydrabamine.

In another embodiment, Formula II is represented by a compound of Formula III:

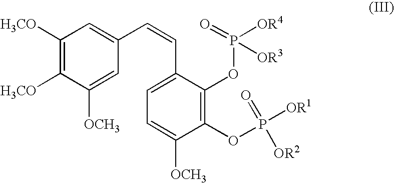

wherein OR¹, OR², OR³ and OR⁴ are each, independently, H, —O⁻QH⁺ or —O⁻M⁺, wherein M⁺ is a monovalent or divalent metal cation, and Q is, independently:

a) an amino acid containing at least two nitrogen atoms where one of the nitrogen atoms, together with a proton, forms a quaternary ammonium cation QH⁺; or

b) an organic containing at least one nitrogen atom which, together with a proton, forms a quaternary ammonium cation, QH⁺.

In one embodiment of Formula III, at least one of OR¹, OR², OR³ and OR⁴ is hydroxyl, and at least one of OR¹, OR², OR³ and OR⁴ is —O⁻QH⁺, where Q is L-histidine. In another embodiment of Formula III, at least one of OR¹, OR², OR³ and OR⁴ is hydroxyl, and at least one of OR¹, OR², OR³ and OR⁴ is TRIS.

### III. RECEPTOR TYROSINE KINASE (RTK) INHIBITORS

In certain aspects of the invention, a VDA is administered together with a kinase inhibitor (e.g., as a single pharmaceutical composition or as separate pharmaceutical compositions). A kinase inhibitor is any drug or agent (e.g., anti-sense; small molecules; antibodies; etc) which blocks or reduces the activity of a kinase. Generally, a “kinase activity” refers to the ability of a polypeptide to catalyze the transfer of a phosphate from one molecule to another.

In exemplary embodiments, the kinase inhibitor is an inhibitor of a receptor tyrosine kinase (RTK). Several RTKs have been associated with cancer, including RTKs which are components of signal transduction pathways that induce abnormal cell growth or inhibit apoptosis in tumor cells. In general, inhibition of aberrant RTK-mediated cell signaling with a RTK inhibitor has been shown to be a feasible anti-cancer therapeutic strategy. Exemplary RTKs associated with cancer include RTKs of the VEGF receptor family (e.g, VEGFR-1, VEGFR-2, VEGFR-3), RTKs of the EGF receptor family, RTKs of the RET receptor family, RTKs of the FGF receptor family (FGFR1, FGFR2, FGFR3) and RTKs of the PDGF receptor family (e.g., PDGFR-alpha, PDGFR-beta).

In certain embodiments, the RTK inhibitor is a multi-kinase inhibitor. For example, in addition to inhibiting an RTK, the inhibitor may also inhibit a serine/threonine kinase implicated in cancer. In certain embodiments, a multi-kinase inhibitor may inhibit multiple kinase targets selected from the group consisting of, but not limited to, e.g., PDGFR-alpha, PDGFR-beta, EGFR, VEGFR, VEGFR1, VEGFR2, VEGFR3, HER-2, KIT, FLT3, C-MET, FGFR, FGFR1, FGFR3, C-FMS, RET, ABL, ALK, ARG, NTRKIm NTRK3, JAK2, ROS, Raf, etc.

In certain embodiments, the RTK inhibitor is a small molecule RTK inhibitor. In one embodiment the small molecule RTK inhibitor is Axitinib (e.g., AG013736, Pfizer, New York, N.Y.). In another embodiment, the small molecule RTK inhibitor is Cediranib (e.g., Recentin® or AZD2171, AstraZeneca, UK). In another embodiment, the small molecule RTK inhibitor is Dasatinib (e.g., Sprycel® or BMS-354825, Bristol-Myers Squibb, Princeton, N.J.). In another embodiment, the small molecule RTK inhibitor is Erlotinib (e.g., Tarceva®, Genentech, San Francisco, Calif. and OSI Pharmaceuticals). In another embodiment, the small molecule RTK inhibitor is Gefitinib (e.g., Iressa®, AstraZeneca, UK). In another embodiment, the small molecule RTK inhibitor is Imatinib (e.g, Imatinib mesilate, Gleevec®, Novartis, Switzerland). In another embodiment, the small molecule RTK inhibitor is Lapatinib (e.g, Lapatinib Ditosylate, GSK572016, GlaxoSmithKline, UK). In another embodiment, the small molecule RTK inhibitor is Nilotinib (e.g., Tasigna®, Novartis, Switzerland). In another embodiment, the small molecule RTK inhibitor is Pazopanib (e.g., GW786034, GlaxoSmithkline, UK). In another embodiment, the small molecule RTK inhibitor is Semaxinib (e.g., SU5416, Pfizer, New York, N.Y.). In another embodiment, the small molecule RTK inhibitor is Sunitinib (e.g., Sutent®, Pfizer, New York, N.Y.). In another embodiment, the small molecule RTK inhibitor is Vatalanib (e.g., PTK787/ZK-222584, Schering AG, Germany). In another embodiment, the small molecule RTK inhibitor is Vandetanib (e.g., ZD6474 or Zactima®, AstraZeneca, UK).

In certain embodiments, the small molecule RTK inhibitor is a compound having formula A-D-B, wherein D is —NH—C(O)—NH—, A is a substituted moiety of up to 40 carbon atoms of the formula: -L-(M-L¹)_{q}, where L is a 5 or 6 membered cyclic structure bound directly to D, L¹ comprises a substituted cyclic moiety having at least 5 members, M is a bridging group having at least one atom, q is an integer of from 1-3; and each cyclic structure of L and L¹ contains 0-4 members of the group consisting of nitrogen, oxygen and sulfur, and B is a substituted or unsubstituted, up to tricyclic aryl or heteroaryl moiety of up to 30 carbon atoms with at least one 6-member cyclic structure bound directly to D containing 0-4 members of the group consisting of nitrogen, oxygen and sulfur, wherein L¹ is substituted by at least one substituent selected from the group consisting of —SO₂Rₓ, —C(O)Rₓ and —C(NR_{y})R_{z}, R_{y} is hydrogen or a carbon based moiety of up to 24 carbon atoms optionally containing heteroatoms selected from N, S and O and optionally halosubstituted, up to per halo, R_{z} is hydrogen or a carbon based moiety of up to 30 carbon atoms optionally containing heteroatoms selected from N, S and O and optionally substituted by halogen, hydroxy and carbon based substituents of up to 24 carbon atoms, which optionally contain heteroatoms selected from N, S and O and are optionally substituted by halogen; Rₓ is R_{z} or NRₐR_{b} where Rₐ and R_{b} are a) independently hydrogen, a carbon based moiety of up to 30 carbon atoms optionally containing heteroatoms selected from N, S and O and optionally substituted by halogen, hydroxy and carbon based substituents of up to 24 carbon atoms, which optionally contain heteroatoms selected from N, S and O and are optionally substituted by halogen, or —OSi(R_{f})₃ where R_{f} is hydrogen or a carbon based moiety of up to 24 carbon atoms optionally containing heteroatoms selected from N, S and O and optionally substituted by halogen, hydroxy and carbon based substituents of up to 24 carbon atoms, which optionally contain heteroatoms selected from N, S and O and are optionally substituted by halogen; or b) Rₐ and R_{b} together form a 5-7 member heterocyclic structure of 1-3 heteroatoms selected from N, S and O, or a substituted 5-7 member heterocyclic structure of 1-3 heteroatoms selected from N, S and O substituted by halogen, hydroxy or carbon based substituents of up to 24 carbon atoms, which optionally contain heteroatoms selected from N, S and O and are optionally substituted by halogen; or c) one of Rₐ or R_{b} is —C(O)—, a C₁-C₅ divalent alkylene group or a substituted C₁-C₅ divalent alkylene group bound to the moiety L to form a cyclic structure with at least 5 members, wherein the substituents of the substituted C₁-C₅ divalent alkylene group are selected from the group consisting of halogen, hydroxy, and carbon based substituents of up to 24 carbon atoms, which optionally contain heteroatoms selected from N, S and O and are optionally substituted by halogen; where B is substituted, L is substituted or L¹ is additionally substituted, the substituents are selected from the group consisting of halogen, up to per-halo, and Wn, where n is 0-3; wherein each W is independently selected from the group consisting of —CN, —CO₂R⁷, —C(O)NR⁷R⁷, —C(O)—R⁷, —NO₂, —OR⁷, —SR⁷, —NR⁷R⁷, —NR⁷C(O)OR⁷, —NR⁷C(O)R⁷, -Q-Ar, and carbon based moieties of up to 24 carbon atoms, optionally containing heteroatoms selected from N, S and O and optionally substituted by one or more substituents independently selected from the group consisting of —CN, —CO₂R⁷, —C(O)R⁷, —C(O)NR⁷R⁷, —OR⁷, —SR⁷, —NR⁷R⁷, —NO₂, —NR⁷C(O)R⁷, —NR⁷C(O)OR⁷ and halogen up to per-halo; with each R⁷ independently selected from H or a carbon based moiety of up to 24 carbon atoms, optionally containing heteroatoms selected from N; S and O and optionally substituted by halogen, wherein Q is —O—, —S—, —N(R⁷)—, —(CH₂)ₘ—, —C(O)—, —CH(OH)—, —(CH₂)ₘO—, —(CH₂)ₘS—, —(CH₂)ₘN(R⁷)—, —O(CH₂)ₘ—CHX^{a}—, —CX^{a}₂—, —S—(CH₂)ₘ— and —N(R⁷)(CH₂)ₘ—, where m=1-3, and X^{a} is halogen; and Ar is a 5- or 6-member aromatic structure containing 0-2 members selected from the group consisting of nitrogen, oxygen and sulfur, which is optionally substituted by halogen, up to per-halo, and optionally substituted by Zₙ₁, wherein n1 is 0 to 3 and each Z is independently selected from the group consisting of —CN, —CO₂R⁷, —C(O)R⁷, —C(O)NR⁷R⁷, —NO₂, —OR⁷, —SR⁷—NR⁷R⁷, —NR⁷C(O)OR⁷, —NR⁷C(O)R⁷, and a carbon based moiety of up to 24 carbon atoms, optionally containing heteroatoms selected from N, S and O and optionally substituted by one or more substituents selected from the group consisting of —CN, —CO₂R⁷, —COR⁷, —C(O)NR⁷R⁷, —OR⁷, —SR⁷, —NO₂, —NR⁷R⁷, —NR⁷C(O)R⁷, and —NR⁷C(O)OR⁷

In certain preferred embodiments, the small-molecule RTK inhibitor is Sorafenib (e.g., Nexavar®, Bayer Pharmaceuticals, Germany)), and pharmaceutically acceptable salts, hydrates, solvates, or polymorphs thereof. A preferred Sorafenib compound is the tosylate salt of the compound N-[4-chloro-3-(trifluoromethyl)-phenyl]-N′-{4-[2-carbamoyl-1-oxo-(4-pyridyloxy)]phenyl}urea:

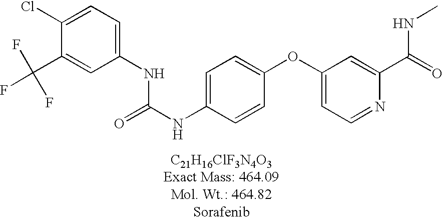

In certain preferred embodiments, the small molecule RTK inhibitor is a compound of Formula IV:

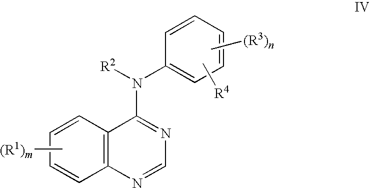

wherein:
m is 1, 2, or 3;
each R¹ is independently selected from the group consisting of hydrogen, halo, hydroxy, hydroxyamino, carboxy, nitro, guanidino, ureido, cyano, trifluoromethyl, and —(C₁-C₄ alkylene)-W-(phenyl) wherein W is a single bond, O, S or NH;
or each R¹ is independently selected from R⁹ and (C₁-C₄)-alkyl substituted by cyano, wherein R⁹ is selected from the group consisting of R⁵, —OR⁶, —NR⁶R⁶, —C(O)R⁷, —NHOR⁵, —OC(O)R⁶, cyano, A and —YR⁵; wherein
   R⁵ is C₁-C₄ alkyl;
   R⁶ is independently hydrogen or R⁵;
   R⁷ is R⁵, —OR⁶ or —NR⁶R⁶;
   A is selected from piperidino, morpholino, pyrrolidino, 4-R⁶-piperazin-1-yl, imidazol-1-yl, 4-pyridon-1-yl, —(C₁-C₄ alkylene)(CO₂H), phenoxy, phenyl, phenylsulfanyl, C₂-C₄ alkenyl, and —(C₁-C₄ alkylene)C(O)NR⁶R⁶; and
   Y is S, SO, or SO₂;
   wherein the alkyl moieties in R⁵, —OR⁶ and —NR⁶R⁶ are optionally substituted by one to three substituents independently selected from halo and R⁹, and
   wherein the alkyl moieties of said optional substituents are optionally substituted by halo or R⁹, with the proviso that two heteroatoms are not attached to the same carbon atom, and with the further proviso that no more than three R⁹ groups may comprise a single R¹ group;
or each R¹ is independently selected from —NHSO₂R⁵, phthalimido-(C₁-C₄)-alkylsulfonylamino, benzamido, benzenesulfonylamino, 3-phenylureido, 2-oxopyrrolidin-1-yl, 2,5-dioxopyrrolidin-1-yl, and R¹⁰—(C₂-C₄)-alkanoylamino wherein R¹⁰ is selected from halo, —OR⁶, C₂-C₄ alkanoyloxy, —C(O)R⁷, and —NR⁶R⁶; and wherein the foregoing R¹ groups are optionally substituted by 1 or 2 substituents independently selected from halo, C₁-C₄ alkyl, cyano, methanesulfonyl and C₁-C₄ alkoxy;
or two R¹ groups are taken together with the carbons to which they are attached to form a 5-8 membered ring that includes 1 or 2 heteroatoms selected from O, S and N;
R² is hydrogen or C₁-C₆ alkyl optionally substituted by 1 to 3 substituents independently selected from halo, C₁-C₄ alkoxy, —NR⁶R⁶, and —SO₂R⁵;
n is 1 or 2;
each R³ is independently selected from hydrogen, halo, hydroxy, C₁-C₆ alkyl, —NR⁶R⁶, and C₁-C₄ alkoxy, wherein the alkyl moieties of said R³ groups are optionally substituted by 1 to 3 substituents independently selected from halo, C₁-C₄ alkoxy, —NR⁶R⁶, and —SO₂R⁵; and
R⁴ is azido or -(ethynyl)-R¹¹ wherein R¹¹ is hydrogen or C₁-C₆ alkyl optionally substituted by hydroxy, —OR⁶, or —NR⁶R⁶.

In other preferred embodiments, the small molecule RTK inhibitor is selected from the group consisting of (6,7-dimethoxyquinazolin-4-yl)-(3-ethynylphenyl)-amine; (6,7-dimethoxyquinazolin-4-yl)-[3-(3′-hydroxypropyn-1-yl)phenyl]-amine; [3-(2′-(aminomethyl)-ethynyl)phenyl]-(6,7-dimethoxyquinazolin-4-yl)-amine; (3-ethynylphenyl)-(6-nitroquinazolin-4-yl)-amine; (6,7-dimethoxyquinazolin-4-yl)-(4-ethynylphenyl)-amine; (6,7-dimethoxyquinazolin-4-yl)-(3-ethynyl-2-methylphenyl)-amine; (6-aminoquinazolin-4-yl)-(3-ethynylphenyl)-amine; (3-ethynylphenyl)-(6-methanesulfonylaminoquinazolin-4-yl)-amine; (3-ethynylphenyl)-(6,7-methylenedioxyquinazolin-4-yl)-amine; (6,7-dimethoxyquinazolin-4-yl)-(3-ethynyl-6-methylphenyl)-amine; (3-ethynylphenyl)-(7-nitroquinazolin-4-yl)-amine; (3-ethynylphenyl)-[6-(4′-toluenesulfonylamino)quinazolin-4-yl]-amine; (3-ethynylphenyl)-{6-[2′-phthalimido-eth-1′-yl-sulfonylamino]quinazolin-4-yl}-amine; (3-ethynylphenyl)-(6-guanidinoquinazolin-4-yl)-amine; (7-aminoquinazolin-4-yl)-(3-ethynylphenyl)-amine; (3-ethynylphenyl)-(7-methoxyquinazolin-4-yl)-amine; (6-carbomethoxyquinazolin-4-yl)-(3-ethynylphenyl)-amine; (7-carbomethoxyquinazolin-4-yl)-(3-ethynylphenyl)-amine; [6,7-bis(2-methoxyethoxy)quinazolin-4-yl]-(3-ethynylphenyl)-amine; (3-azidophenyl)-(6,7-dimethoxyquinazolin-4-yl)-amine; (3-azido-5-chlorophenyl)-(6,7-dimethoxyquinazolin-4-yl)-amine; (4-azidophenyl)-(6,7-dimethoxyquinazolin-4-yl)-amine; (3-ethynylphenyl)-(6-methansulfonyl-quinazolin-4-yl)-amine; (6-ethansulfanyl-quinazolin-4-yl)-(3-ethynylphenyl)-amine; (6,7-dimethoxy-quinazolin-4-yl)-(3-ethynyl-4-fluoro-phenyl)-amine; (6,7-dimethoxy-quinazolin-4-yl)-[3-(propyn-1′-yl)-phenyl]-amine; [6,7-bis-(2-methoxy-ethoxy)-quinazolin-4-yl]-(5-ethynyl-2-methyl-phenyl)-amine; [6,7-bis-(2-methoxy-ethoxy)-quinazolin-4-yl]-(3-ethynyl-4-fluoro-phenyl)-amine; [6,7-bis-(2-chloro-ethoxy)-quinazolin-4-yl]-(3-ethynyl-phenyl)-amine; [6-(2-chloro-ethoxy)-7-(2-methoxy-ethoxy)-quinazolin-4-yl]-(3-ethynyl-phenyl)-amine; [6,7-bis-(2-acetoxy-ethoxy)-quinazolin-4-yl]-(3-ethynyl-phenyl)-amine; 2-[4-(3-ethynyl-phenylamino)-7-(2-hydroxy-ethoxy)-quinazolin-6-yloxy]-ethanol; [6-(2-acetoxy-ethoxy)-7-(2-methoxy-ethoxy)-quinazolin-4-yl]-(3-ethynyl-phenyl)-amine; [7-(2-chloro-ethoxy)-6-(2-methoxy-ethoxy)-quinazolin-4-yl]-(3-ethynyl-phenyl)-amine; [7-(2-acetoxy-ethoxy)-6-(2-methoxy-ethoxy)-quinazolin-4-yl]-(3-ethynyl-phenyl)-amine; 2-[4-(3-ethynyl-phenylamino)-6-(2-hydroxy-ethoxy)-quinazolin-7-yloxy]-ethanol; 2-[4-(3-ethynyl-phenylamino)-7-(2-methoxy-ethoxy)-quinazolin-6-yloxy]-ethanol; 2-[4-(3-ethynyl-phenylamino)-6-(2-methoxy-ethoxy)-quinazolin-7-yloxy]-ethanol; [6-(2-acetoxy-ethoxy)-7-(2-methoxy-ethoxy)-quinazolin-4-yl]-(3-ethynyl-phenyl)-amine; (3-ethynyl-phenyl)-{6-(2-methoxy-ethoxy)-7-[2-(4-methyl-piperazin-1-yl)-ethoxy]-quinazolin-4-yl}-amine; (3-ethynyl-phenyl)-[7-(2-methoxy-ethoxy)-6-(2-morpholin-4-yl)-ethoxy)-quinazolin-4-yl]-amine; (6,7-diethoxyquinazolin-1-yl)-(3-ethynylphenyl)-amine; (6,7-dibutoxyquinazolin-1-yl)-(3-ethynylphenyl)-amine; (6,7-diisopropoxyquinazolin-1-yl)-(3-ethynylphenyl)-amine; (6,7-diethoxyquinazolin-1-yl)-(3-ethynyl-2-methyl-phenyl)-amine; [6,7-bis-(2-methoxy-ethoxy)-quinazolin-1-yl]-(3-ethynyl-2-methyl-phenyl)-amine; (3-ethynylphenyl)-[6-(2-hydroxy-ethoxy)-7-(2-methoxy-ethoxy)-quinazolin-1-yl]-amine; [6,7-bis-(2-hydroxy-ethoxy)-quinazolin-1-yl]-(3-ethynylphenyl)-amine; 2-[4-(3-ethynyl-phenylamino)-6-(2-methoxy-ethoxy)-quinazolin-7-yloxy]-ethanol; (6,7-dipropoxy-quinazolin-4-yl)-(3-ethynyl-phenyl)-amine; (6,7-diethoxy-quinazolin-4-yl)-(3-ethynyl-5-fluoro-phenyl)-amine; (6,7-diethoxy-quinazolin-4-yl)-(3-ethynyl-4-fluoro-phenyl)-amine; (6,7-diethoxy-quinazolin-4-yl)-(5-ethynyl-2-methyl-phenyl)-amine; (6,7-diethoxy-quinazolin-4-yl)-(3-ethynyl-4-methyl-phenyl)-amine; (6-aminomethyl-7-methoxy-quinazolin-4-yl)-(3-ethynyl-phenyl)-amine; (6-aminomethyl-7-methoxy-quinazolin-4-yl)-(3-ethynylphenyl)-amine; (6-aminocarbonylmethyl-7-methoxy-quinazolin-4-yl)-(3-ethynylphenyl)-amine; (6-aminocarbonylethyl-7-methoxy-quinazolin-4-yl)-(3-ethynylphenyl)-amine; (6-aminocarbonylmethyl-7-ethoxy-quinazolin-4-yl)-(3-ethynylphenyl)-amine; (6-aminocarbonylethyl-7-ethoxy-quinazolin-4-yl)-(3-ethynylphenyl)-amine; (6-aminocarbonylmethyl-7-isopropoxy-quinazolin-4-yl)-(3-ethynylphenyl)-amine; (6-aminocarbonylmethyl-7-propoxy-quinazolin-4-yl)-(3-ethynylphenyl)-amine; (6-aminocarbonylmethyl-7-methoxy-quinazolin-4-yl)-(3-ethynylphenyl)-amine; (6-aminocarbonylethyl-7-isopropoxy-quinazolin-4-yl)-(3-ethynylphenyl)-amine; (6-aminocarbonylethyl-7-propoxy-quinazolin-4-yl)-(3-ethynylphenyl)-amine; (6,7-diethoxyquinazolin-1-yl)-(3-ethynylphenyl)-amine; (3-ethynylphenyl)-[6-(2-hydroxy-ethoxy)-7-(2-methoxy-ethoxy)-quinazolin-1-yl]-amine; [6,7-bis-(2-hydroxy-ethoxy)-quinazolin-1-yl]-(3-ethynylphenyl)-amine; [6,7-bis-(2-methoxy-ethoxy)-quinazolin-1-yl]-(3-ethynylphenyl)-amine; (6,7-dimethoxyquinazolin-1-yl)-(3-ethynylphenyl)-amine; (3-ethynylphenyl)-(6-methanesulfonylamino-quinazolin-1-yl)-amine; and (6-amino-quinazolin-1-yl)-(3-ethynylphenyl)-amine.

In certain preferred embodiments, the small-molecule RTK inhibitor is Erlotinib (e.g., Tarceva®, Genentech, San Francisco, Calif. and OSI Pharmaceuticals), and pharmaceutically acceptable salts, hydrates, solvates, or polymorphs thereof. A preferred Erlotinib compound is the hydrochloride salt of the compound N-(3-ethynylphenyl)-6,7-bis(2-methoxyethoxy)-4-quinazolinamine:

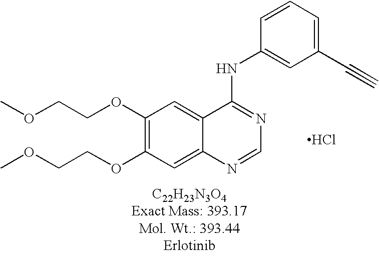

Examples of other kinase inhibitors useful in the methods of the invention, include, but are not limited to, e.g., 17-DMAG; 17-AAG; AG 9; AG 10; AG 1; AG 18; AG 30; AG 43; AG 82; AG 99; AG 112; AG 126; AG 183; AG 213; AG 370: AG 490; AG 494; AG 527; AG 537; AG 538; AG 555; AG 556; AG 592; AG 825; AG 835; AG 879; AG 957; AG 957; AG 1024; AG 1288; AG 1295; AG 1296; AG 1387; AG 1433; AG 1478; AGL 2043; AGL 2263; Aminogenistein; ABX-EGF, adaphostin, AEE788, AG 013736, AG 490, AG 825, AG 957, AG 1024, AG 1296, aloisine, aloisine A, alsterpaullone, aminogenistein, AMG 706, AMN107, API-2, AP23573, apigenin, ARRY-142886 (AZD6244), arctigenin, AY-22989, AZD0530, AZD1 152, AZD2171, bisindolylmaleimide IX, BMS-354825, BMS-387032, BMS-599626, Bryostatin 1, BPDQ; BPIQ-I; BPIQ-II; Butein; Cucurbitacin I, Cucumis sativus L.; Curcumin, Curcuma longa L.; CC1779, CEP-701, CEP-7055, 2C4, chelerythrine, CHIR-258, CI-1033, CPT-1 1, CP724714, CGP52421, CP-547-632, CT52923, CYC202, D816X, Daidzein; Damnacanthal; Daphnetin; DMBI; Emodin; edelfosine, erbstatin analog, ET18OCH3, everolimus (RAD0001), fasudil, FK506, Geldanamycin (Streptomyces hygroscopicus); Genistein; Genistin; GTP-14564, GO 6976, GW2974, GW572016, GW786034, H-7, H-8, H-89, HA-100, HA-1004, HA-1077, HA-1 100, Herbimycin A; hydroxyfasudil, Isis 3521, indirubin-3′-oxime, 5-Iodotubercidin, kenpaullone, KN-62, KY12420, Lavendustin A; Lavendustin B; LFM-A1 1; LFM-A12; LFM-A13, limofosine, luteolin, LY294002, LY294002, LY333531, LY379196, mallotoxin, midostaurin, ML-9, MLN518, NSC-154020, NSC-226080, NSC-664704, NSC-680410, NU6102, olomoucine, oxindole I PD 0173074, PD 0325901, PD 153035, PD 98059, PD 169316, PD 184352, phloridzin, Perifosine, PKC412, piceatannol, picropodophyllin, PP1, PP2, purvalanol A1, quercetin, RAPA, rapamune, rapamycin, RO 318220, RO 320432, roscovitine, rottlerin, SB202190, SB203580, sirolimus, SL327, SMS-354825, SP600125, staurosporine, STI-571, ST638; SU11652; SU4984; SU5614; SU6656; SU101, SU1498, SU4312, SU6656, SU5402, SU5416, SU6668, SU11248, syk inhibitor, TBB, TCN, Triciribine, Tyrphostin AG 490, Tyrphostin AG 825, Tyrphostin AG 957, Tyrphostin AG 1024, wortmannin, XL184, XL647, XL999, Y-27632, U0126, UCN-01, VX-680, WHI-P97; WHI-P154; WHI-P131; ZM 252868; ZM323881; ZM 39923; and ZM 449829, and analogs and derivatives thereof, etc.

Other RTK inhibitors that may be used to practice the methods of the invention include but are not limited to linear peptides, cyclic peptides, natural amino-acids, unnatural amino acids, and peptidomimetic compounds; anti-RTK siRNAs; anti-RTK antibodies (e.g., bevacizumab (Avastin®, Genentech, San Francisco, Calif.), cetuximab (Erbitbux®, Imclone Systems, New York, N.Y.), or trastuzumab (Herceptin®, Genentech, San Francisco, Calif.).

### IV. PREFERRED DOSAGE RANGES

### Two-Component Combination Therapy

In accordance with the present invention, improved, two-component chemotherapeutic regimens comprising a VDA (e.g., a combretastatin) and a RTK inhibitor are provided for the treatment of cancer. The improved chemotherapeutic regimens can enhance efficacy for the treatment of neoplastic disease. For example, the present methods permit a clinician to administer a combretastatin compound, and a small molecule RTK inhibitor, at dosages which are significantly lower than those employed for the single agent. Preferred dosages suitable for administration of a small molecule RTK inhibitor and a combretastatin compound in accordance with the invention are set forth herein below. Whether administered simultaneously or sequentially, the combretastatin compound and the at RTK inhibitor can be administered in any amount or by any route of administration effective for the modulation of tumor growth or metastasis, especially treatment of cancer as described herein.

In one exemplary embodiment, a combretastatin prodrug (e.g. CA4P or CA1P) is administered together with Sorafenib. In a particularly preferred embodiment, a pharmaceutical composition comprising Sorafenib and CA1P are used to treat cancer in a subject, wherein the subject is human.

In another exemplary embodiment, a combretastatin prodrug (e.g. CA4P or CA1P) is administered together with Erlotinib. In a particularly preferred embodiment, a pharmaceutical composition comprising Erlotinib and CA1P are used to treat cancer in a subject, wherein the subject is human.

A suitable dose per day for each of the compounds, i.e., a small molecule RTK inhibitor (e.g., Sorafenib or Erlotinib), and a VDA (e.g. a combretastatin, e.g., CA4P or CA1P), can be, individually, in the range of from about 1 ng to about 10,000 mg, about 5 ng to about 9,500 mg, about 10 ng to about 9,000 mg, about 20 ng to about 8,500 mg, about 30 ng to about 7,500 mg, about 40 ng to about 7,000 mg, about 50 ng to about 6,500 mg, about 100 ng to about 6,000 mg, about 200 ng to about 5,500 mg, about 300 ng to about 5,000 mg, about 400 ng to about 4,500 mg, about 500 ng to about 4,000 mg, about 1 μg to about 3,500 mg, about 5 μg to about 3,000 mg, about 10 μg to about 2,600 mg, about 20 μg to about 2,575 mg, about 30 μg to about 2,550 mg, about 40 μg to about 2,500 mg, about 50 μg to about 2,475 mg, about 100 μg to about 2,450 mg, about 200 μg to about 2,425 mg, about 300 μg to about 2,000, about 400 μg to about 1,175 mg, about 500 μg to about 1,150 mg, about 0.5 mg to about 1,125 mg, about 1 mg to about 1,100 mg, about 1.25 mg to about 1,075 mg, about 1.5 mg to about 1,050 mg, about 2.0 mg to about 1,025 mg, about 2.5 mg to about 1,000 mg, about 3.0 mg to about 975 mg, about 3.5 mg to about 950 mg, about 4.0 mg to about 925 mg, about 4.5 mg to about 900 mg, about 5 mg to about 875 mg, about 10 mg to about 850 mg, about 20 mg to about 825 mg, about 30 mg to about 800 mg, about 40 mg to about 775 mg, about 50 mg to about 750 mg, about 100 mg to about 725 mg, about 200 mg to about 700 mg, about 300 mg to about 675 mg, about 400 mg to about 650 mg, about 500 mg, or about 525 mg to about 625 mg.

Other suitable doses for the compounds of the invention include, for example, 0.1 mg/kg to about 100 mg/kg; from about 1 mg/kg to about 100 mg/kg; from about 5 mg/kg to about 50 mg/kg; from about 10 to about 25 mg/kg; about 10 mg/kg; about 15 mg/kg; about 20 mg/kg; about 25 mg/kg; about 30 mg/kg; about 40 mg/kg; about 50 mg/kg; about 60 mg/kg; about 70 mg/kg; about 80 mg/kg; about 90 mg/kg; and about 100 mg/kg.

### V. MULTI-COMPONENT COMBINATION THERAPY

In certain aspects, the combination therapy methods and pharmaceutical compositions of the invention may comprise other anticancer agents in addition to a VDA and RTK inhibitor. As explained above, numerous types of anticancer agents are exemplary of those having applications in a combination therapy with the pharmaceutical compositions (e.g., Sorafenib or Erlotinib and CA1P or CA4P) and methods of the present invention. Such classes of anticancer agents, and their preferred mechanisms of action, are described below:

1. Alkylating agent: a compound that donates an alkyl group to nucleotides. Alkylated DNA is unable to replicate itself and cell proliferation is stopped. Examples of such compounds include, but are not limited to, busulfan (Myleran®), coordination metal complexes (e.g. platinum coordination compounds such as carboplatin, oxaliplatin, and cisplatin), cyclophosphamide (Cytoxan®), dacarbazine, ifosfamide, lomustine, procarbazine, mechlorethamine (mustargen), and melphalan;

2. Bifunctional alkylating agent: a compound having two labile methanesulfonate groups that are attached to opposite ends of a four carbon alkyl chain. The methanesulfonate groups interact with, and cause damage to DNA in cancer cells, preventing their replication. Examples of such compounds include, without limitation, chlorambucil and melphalan;

3. Non-steroidal aromatase inhibitor: a compound that inhibits the enzyme aromatase, which is involved in estrogen production. Thus, blockage of aromatase results in the prevention of the production of estrogen. Examples of such compounds include anastrozole and exemstane;

4. Immunotherapeutic agent: an antibody or antibody fragment which targets cancer cells that produce proteins associated with malignancy. Exemplary immunotherapeutic agents include Herceptin® (Genentech, South San Francisco, Calif.) which targets HER2 or HER2/neu, which occurs in high numbers in about 25 percent to 30 percent of breast cancers; Erbitux® (ImClone Systems, New York, N.Y.) which targets the Epidermal Growth Factor Receptor (EGFR) in colon cancers; Avastin® (Genentech, South San Francisco, Calif.) which targets the Vascular Endothelial Growth Factor (VEGF) expressed by colon cancers; and rituximab (Rituxan®, Genentech, South San Francisco, Calif.) an anti-CD20 antibody which triggers apoptosis in B cell lymphomas. Additional immunotherapeutic agents include immunotoxins, wherein toxin molecules such as ricin, diphtheria toxin and pseudomonas toxins are conjugated to antibodies which recognize tumor specific antigens. Conjugation can be achieved biochemically or via recombinant DNA methods.

5. Nitrosurea compound: inhibits enzymes that are needed for DNA repair. These agents are able to travel to the brain so they are used to treat brain tumors, as well as non-Hodgkin's lymphomas, multiple myeloma, and malignant melanoma. Examples of nitrosureas include carmustine and lomustine;

6. Antimetabolite: a class of drugs that interfere with DNA and ribonucleic acid (RNA) synthesis. These agents are phase specific (S phase) and are used to treat chronic leukemias as well as tumors of breast, ovary and the gastrointestinal tract. Examples of antimetabolites include 5-fluorouracil, 6-thioguanine, 6-mercaptopurine, 5-azacytidine, cladribine, fludarabine, hydroxyurea, methotrexate, gemcitabine (GEMZAR®), cytarabine (cytosine arabinoside, Ara-C, Cytosar-U), and fludarabine.

7. Antitumor antibiotic: a compound having antimicrobial and cytotoxic activity. Such compounds also may interfere with DNA by chemically inhibiting enzymes and mitosis or altering cellular membranes. Examples include, but certainly are not limited to bleomycin, dactinomycin, daunorubicin, doxorubicin (Adriamycin), idarubicin, and the manumycins (e.g. Manumycins A, C, D, E, and G and their derivatives; see for example U.S. Pat. No. 5,444,087);

8. Mitotic inhibitor: a compound that can inhibit mitosis (e.g., tubulin binding compounds) or inhibit enzymes that prevent protein synthesis needed for reproduction of the cell. Examples of mitotic inhibitors include taxanes such as paclitaxel and docetaxel, epothilones, etoposide, vinblastine, vincristine, and vinorelbine.

9. Radiation therapy: includes but is not limited to X-rays or gamma rays which are delivered from either an externally supplied source such as a beam or by implantation of small radioactive sources.

10. Topoisomerase I inhibitors: agents which interfere with topoisomerase activity thereby inhibiting DNA replication. Such agents include, without limitation, CPT-11 and topotecan.

11. Hormonal therapy: includes, but is not limited to anti-estrogens, such as Tamoxifen, GnRH agonists, such as Lupron, and Progestin agents, such as Megace.

Naturally, other types of anticancer agents that function via a large variety of mechanisms have combination therapy application in the pharmaceutical compositions and methods of the present invention. Additional such agents include for example, leuocovorin, kinase inhibitors, such as Iressa and Flavopiridol, analogues of conventional chemotherapeutic agents such as taxane analogs and epothilone analogues, antiangiogenics such as matrix metalloproteinase inhibitors, and other VEGF inhibitors, such as Bevacizumab (Genentech, South San Francisco, Calif.). ZD6474 and SU6668. Retinoids such as Targretin can also be employed in the pharmaceutical compositions and methods of the invention. Signal transduction inhibitors which interfere with farnesyl transferase activity and chemotherapy resistance modulators, e.g., Valspodar can also be employed. Monoclonal antibodies such as C225 and anti-VEGFr antibodies can also be employed.

### VI. PHARMACEUTICAL COMPOSITIONS

As explained above, the present methods can, for example, be carried out using a single pharmaceutical composition comprising both a VDA and a RTK inhibitor when administration is to be simultaneous or sequential. Alternatively, the VDA and RTK inhibitor may be administered simultaneously or sequentially as separate pharmaceutical compositions.

Pharmaceutical compositions employed in the methods of the invention include a compound (e.g., a VDA and/or RTK inhibitor) formulated with other ingredients, e.g., “pharmaceutically acceptable carriers”. Preferably, as used herein, the term “pharmaceutically acceptable” means approved by a regulatory agency of the Federal or a state government or listed in the U.S. Pharmacopeia or other generally recognized pharmacopeia for use in animals, and more particularly in humans. The term “carrier” refers, for example to a diluent, adjuvant, excipient, auxiliary agent or vehicle with which an active agent of the present invention is administered. Such pharmaceutical carriers can be sterile liquids, such as water and oils, including those of petroleum, animal, vegetable or synthetic origin, such as peanut oil, soybean oil, mineral oil, sesame oil and the like. Water or aqueous saline solutions and aqueous dextrose and glycerol solutions are preferably employed as carriers, particularly for injectable solutions. Other pharmaceutical carriers include, but are not limited to, antioxidants, preservatives, dyes, tablet-coating compositions, plasticizers, inert carriers, excipients, polymers, coating materials, osmotic barriers, devices and agents which slow or retard solubility, etc. Non-toxic pharmaceutically acceptable excipients that are suitable for the manufacture of tablets include, for example, inert diluents, such as calcium carbonate, sodium carbonate, lactose, calcium phosphate or sodium phosphate; granulating and disintegrating agents, for example, corn starch, or alginic acid; and binding agents, for example magnesium stearate, stearic acid or talc. Suitable pharmaceutical carriers are described in “Remington's Pharmaceutical Sciences” by E. W. Martin.

A pharmaceutical composition of the present invention can be administered by any suitable route, for example, by injection, by oral, pulmonary, nasal or other forms of administration. In general, pharmaceutical compositions contemplated to be within the scope of the invention, comprise, inter alia, pharmaceutically acceptable diluents, preservatives, solubilizers, emulsifiers, adjuvants and/or carriers. Such compositions can include diluents of various buffer content (e.g., Tris-HCl, acetate, phosphate), pH and ionic strength; additives such as detergents and solubilizing agents (e.g., Tween 80, Polysorbate 80), anti-oxidants (e.g., ascorbic acid, sodium metabisulfite), preservatives (e.g., Thimersol, benzyl alcohol) and bulking substances (e.g., lactose, mannitol); incorporation of the material into particulate preparations of polymeric compounds such as polylactic acid, polyglycolic acid, etc., or into liposomes. Such compositions may influence the physical state, stability, rate of in vivo release, and rate of in vivo clearance of components of a pharmaceutical composition of the present invention. See, e.g., Remington's Pharmaceutical Sciences, 18th Ed. (1990, Mack Publishing Co., Easton, Pa. 18042) pages 1435-1712 which are herein incorporated by reference. A pharmaceutical composition of the present invention can be prepared, for example, in liquid form, or can be in dried powder, such as lyophilized form. Particular methods of administering such compositions are described infra.

Suitable pharmaceutical compositions for oral use, include, but are not limited to, tablets, troches, lozenges, aqueous or oily suspensions, dispersible powders or granules, emulsions, hard or soft capsules, solutions, syrups and elixirs. Compositions intended for oral use may be prepared according to any method known to the art for the manufacture of pharmaceutical compositions. Such compositions may contain one or more agents selected from the group consisting of diluents, sweetening agents, flavoring agents, coloring agents and preserving agents in order to provide palatable preparations. Formulations for oral use may also be presented as hard gelatin capsules wherein the active ingredient is mixed with an inert solid diluent, for example, calcium carbonate, calcium phosphate or kaolin, or as soft gelatin capsules wherein the active ingredient is mixed with water or an oil medium, for example peanut oil, liquid paraffin or olive oil.

Aqueous suspensions containing the active materials in admixture with excipients suitable for the manufacture of aqueous suspensions may also be used. Such excipients are suspending agents, for example sodium carboxymethylcellulose, methylcellulose, hydroxypropyl-methylcellulose, sodium alginate, polyvinylpyrrolidone, gum tragacanth and gum acacia; dispersing or wetting agents may be a naturally-occurring phosphatide, for example, lecithin, or condensation products of an alkylene oxide with fatty acids, for example polyoxyethylene stearate, or condensation products of ethylene oxide with long chain aliphatic alcohols, for example heptadecaethylene oxycetanol, or condensation products of ethylene oxide with partial esters derived from fatty acids and hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more preservatives, for example ethyl, or n-propyl hydroxybenzoate, one or more coloring agents, one or more flavoring agents, and one or more sweetening agents, such as sucrose or saccharin.

Dispersible powders and granules suitable for preparation of an aqueous suspension by the addition of water provide the active ingredient in admixture with a dispersing or wetting agent, suspending agent and one or more preservatives. Suitable dispersing or wetting agents and suspending agents are exemplified by those already mentioned above. Additional excipients, for example, sweetening, flavoring and coloring agents, may also be present.

The compounds of the invention may also be in the form of non-aqueous liquid formulations, e.g., oily suspensions which may be formulated by suspending the active ingredients in a vegetable oil, for example arachis oil, olive oil, sesame oil or peanut oil, or in a mineral oil such as liquid paraffin. The oily suspensions may contain a thickening agent, for example beeswax, hard paraffin or cetyl alcohol. Sweetening agents such as those set forth above, and flavoring agents may be added to provide palatable oral preparations. These compositions may be preserved by the addition of an anti-oxidant such as ascorbic acid.

Pharmaceutical compositions of the invention may also be in the form of oil-in-water emulsions. The oily phase may be a vegetable oil, for example olive oil or arachis oil, or a mineral oil, for example liquid paraffin or mixtures of these. Suitable emulsifying agents may be naturally-occurring gums, for example gum acacia or gum tragacanth, naturally-occurring phosphatides, for example soy bean, lecithin, and esters or partial esters derived from fatty acids and hexitol anhydrides, for example sorbitan monooleate, and condensation products of the said partial esters with ethylene oxide, for example polyoxyethylene sorbitan monooleate. The emulsions may also contain sweetening and flavoring agents.

Syrups and elixirs may be formulated with sweetening agents, for example glycerol, propylene glycol, sorbitol or sucrose. Such formulations may also contain a demulcent, a preservative and flavoring and coloring agents.

The compounds of the invention may also be administered in the form of suppositories for rectal or vaginal administration of the drug. These compositions can be prepared by mixing the drug with a suitable non-irritating excipient which is solid at ordinary temperatures but liquid at the rectal temperature or vaginal temperature and will therefore melt in the rectum or vagina to release the drug. Such materials include cocoa butter and polyethylene glycols.

### VII. DISEASE INDICATIONS

Diseases which can be treated in accordance with present invention include, but are not limited: Accelerated Phase Chronic Myelogenous Leukemia; Acute Erythroid Leukemia; Acute Lymphoblastic Leukemia; Acute Lymphoblastic Leukemia in Remission; Acute Lymphocytic Leukemia; Acute Monoblastic and Acute Monocytic Leukemia; Acute Myelogenous Leukemia; Acute Myeloid Leukemia; Adenocarcinoma; Adenocarcinoma of the Colon; Adenocarcinoma of the Esophagus; Adenocarcinoma of the Lung; Adenocarcinoma of the Pancreas; Adenocarcinoma of the Prostate; Adenocarcinoma of the Rectum; Adenocarcinoma of the Stomach; Adenoid Cystic Carcinoma of the Head and Neck; Adenosquamous Cell Lung Cancer; Adult Giant Cell Glioblastoma; Advanced Adult Primary Liver Cancer; Advanced Gastrointestinal Stromal Tumor; Advanced Non-Nasopharyngeal Head and Neck Carcinoma; Advanced NSCLC; Advanced Solid Tumors; Agnogenic Myeloid; Metaplasia; Anaplastic Astrocytoma; Anaplastic Oligodendroglioma; Anaplastic Thyroid Cancer; Astrocytoma; Atypical Chronic Myelogenous Leukemia; B-Cell Adult Acute Lymphoblastic Leukemia; Basal Cell Carcinoma; Bladder Cancer; Blastic Phase Chronic Myelogenous Leukemia; Bone Metastases; Brain Tumor; Breast Cancer; Breast Cancer in Situ; Breast Neoplasms; Brenner Tumor; Bronchoalveolar Cell Lung Cancer; Cancer of the Fallopian Tube; Carcinoma, Squamous Cell; Central Nervous System Cancer; Cervix Neoplasms; Childhood Acute Lymphoblastic Leukemia; Childhood Acute Lymphoblastic Leukemia in Remission; Childhood Brain Tumor; Childhood Central Nervous System Germ Cell Tumor; Childhood Cerebellar Astrocytoma; Childhood Chronic Myelogenous Leukemia; Childhood Ependymoma; Childhood Malignat Germ Cell Tumor; Childhood Oligodendroglioma; Childhood Soft Tissue Sarcoma; Chondrosarcoma; Chordoma; Chronic Eosinophilic Leukemia (CEL); Chronic Idiopathic Myelofibrosis; Chronic Myelogenous Leukemia; Chronic Myeloid Leukemia; Chronic Myelomonocytic Leukemia; Chronic Phase Chronic Myelogenous Leukemia; Colon Cancer; Colorectal Cancer; Congenital Fibrosarcoma; Dermatofibrosarcoma; Dermatoftbrosarcoma Protuberans (DFSP); Desmoid Tumor; Endometrial Adenocarcinoma; Endometrial Adenosquamous Cell; Eosinophilia; Esophageal Cancer; Epidemic Kaposi's Sarcoma; Epithelial Mesothelioma; Esophageal Cancer; Esophagogastric Cancer; Essential Thrombocythemia; Ewing's Family of Tumors; Extensive Stage Small Cell Lung Cancer; Extrahepatic Bile Duct Cancer; Fallopian Tube Cancer; Familiar Hypereosinophilia; Fibrosarcoma; Follicular Thyroid Cancer; Gallbladder Cancer; Gastric Adenocarcinoma; Gastric Cancer; Gastroinstestinal Cancer; Gastrinoma; Gastrointestinal Carcinoid; Gastrointestinal Neoplasm; Gastrointestinal Stromal Tumor; Giant Cell Glioblastoma; Glioblastoma; Glioma; Glioblastoma Multiforme; Gliosarcoma; Grade I Meningioma; Grade II Meningioma; Grade III Meningioma; Head and Neck Cancer; Head and Neck Neoplasms; Hematopoietic and Lymphoid Cancer, Hepatocellular Carcinoma; High-Grade Childhood Cerebral Astrocytoma; Hypereosinophilic Syndrome; Hypopharyngeal Cancer; Idiopathic Pulmonary Fibrosis; Inflammatory Myofibroblastic Tumor; Inoperable Locally Advanced Squamous Cell Carcinoma of Head and Neck; Insulinoma; Intraductal Breast Carcinoma; Islet Cell Carcinoma; Kidney and Urinary Cancer; L1 Adult Acute Lymphoblastic Leukemia; L2 Adult Acute Lymphoblastic Leukemia; Large Cell Lung Cancer; Laryngeal Cancer; Leukemia, Lymphocytic, Acute L2; Leukemia, Myeloid, Chronic; Leukemia, Myeloid, Chronic Phase; Lip and Oral Cavity Cancer; Lip Cancer; Liposarcoma; Liver Cancer; Liver Dysfunction and Neoplasm; Localized Unresectable Adult Primary Liver Cancer; Low-Grade Childhood Cerebral Astrocytoma; Lymphoid Blastic Phase of Chronic Myeloid Leukemia; Lung Adenocarcinoma With Bronchiole-Alveolar Feature; Lung Cancer; Male Breast Cancer; Malignant Fibrous Histiocytoma; Malignant Melanoma; Mastocytosis; Medullary Thyroid Cancer; Melanoma; Meningeal Tumors; Meningeal Hemangiopericytoma; Meningioma; Meningioma; Meningioma; Mesothelioma; Metastatic Cancer; Metastatic Solid Tumors; Metastatic Colorectal Cancer; Metastatic Gastrointestinal Carcinoid Tumor; Metastatic Pancreatic Carcinoma; Mixed Gliomas; Multiple Myeloma; Musculoskeletal Tumors; Myelodysplastic Syndrome; Myelogenous Leukemia, Acute; Myelofibrosis; Myeloid Leukemia, Chronic; Myeloid Leukemia, Chronic Accelerated-Phase; Myeloid Leukemia, Chronic, Chronic-Phase; Myeloid Metaplasia; Myeloproliferative Disorder (MPD) with Eosinophilia; Myxosarcoma; Nasopharyngeal Cancer; Nasopharyngeal Carcinoma; Neoplasms; Neuroblastoma; Neurofibrosarcoma; Non-B Childhood Acute Lymphoblastic Leukemia; Non-Metastatic (T2-T4, N0-N3, MO; Stages I1 and III) and Histologically-Confirmed Intestinal GC; Non-Metastatic Prostate Cancer; Nonresectable Adrenocortical Carcinoma; Non-Small Cell Lung Cancer; Nose Cancer; Oligodendroglioma; Oligodendroglial Tumors; Oral Cancer; Oropharyngeal Cancer; Osteosarcoma; Osteogenic Sarcoma; Ovarian Cancer; Ovarian Epithelial Cancer; Ovarian Germ Cell Tumor; Ovarian Low Malignant Potential Tumor; Ovarian Neoplasms; Pancreatic Cancer; Papillary Thyroid Cancer; Pelvic Neoplasms; Peritoneal Cavity Cancer; Peritoneal Carcinoma; Peritoneal Neoplasms; Pharynx Cancer; Philadelphia Chromosome Positive Chronic Myelogenous Leukemia; Philadelphia Positive Acute Lymphoblastic Leukemia; Philadelphia Positive Chronic Myeloid Leukemia in Myeloid Blast Crisis; Pneumonic-Type Adenocarcinoma (P-ADC); Polycythemia Vera; Pulmonary Fibrosis; Primary Hepatocellular Carcinoma; Primary Liver Cancer; Prostate Cancer; Prostate Cancer, Antigen Independent; Rectal Cancer; Recurrent Adult Brain Tumor; Recurrent Adult Soft Tissue Sarcoma; Recurrent Adult Primary Liver Cancer; Recurrent Breast Cancer; Recurrent Cervical Cancer; Recurrent Colon Cancer; Recurrent Endometrial Cancer, Recurrent Esophageal Cancer; Recurrent Gastric Cancer; Recurrent Glioblastoma; Recurrent Glioblastoma Multiforme (GBM); Recurrent Kaposi's Sarcoma; Recurrent Melanoma; Recurrent Merkel Cell Carcinoma; Recurrent Ovarian Epithelial Cancer; Recurrent Pancreatic Cancer; Recurrent Prostate Cancer; Recurrent Rectal Cancer; Recurrent Salivary Gland Cancer; Recurrent Skin Cancer; Recurrent Small Cell Lung Cancer; Recurrent Tumors of the Ewing's Family; Recurrent Uterine Sarcoma; Refractory Germ Cell Tumors Expressing EGRF; Relapsing Chronic Myelogenous Leukemia; Renal Cell Cancer; Renal Cell Carcinoma; Renal Papillary Carcinoma; Rhabdomyosarcomas; Salivary Gland Adenoid Cystic Carcinoma; Sarcoma; Sarcomatous Mesothelioma; Skin Cancer; Small Cell Lung Cancer; Soft Tissue Sarcoma; Squamous Cell Carcinoma; Squamous Cell Carcinoma of the Esophagus; Squamous Cell Carcinoma of the Head and Neck; Squamous Cell Carcinoma of the Skin; Squamous Cell Lung Cancer; Stage II Esophageal Cancer; Stage III Esophageal Cancer, Stage II Melanoma; Stage II Merkel Cell Carcinoma; Stage III Adult Soft Tissue Sarcoma; Stage III Esophageal Cancer; Stage III Merkel Cell Carcinoma; Stage III Ovarian Epithelial Cancer; Stage III Pancreatic Cancer; Stage III Salivary Gland Cancer; Stage IIIB Breast Cancer; Stage IIIC Breast Cancer; Stage IV Adult Soft Tissue Sarcoma; Stage IV Breast Cancer; Stage IV Colon Cancer; Stage IV Esophageal Cancer; Stage IV Gastric Cancer; Stage IV Melanoma; Stage IV Ovarian Epithelial Cancer; Stage IV Prostate Cancer; Stage IV Rectal Cancer; Stage IV Salivary Gland Cancer; Stage IVA Pancreatic Cancer; Stage IVB Pancreatic Cancer; Sweat Gland Carcinoma; Sebaceous Gland Carcinoma; Systemic Mastocytosis; Synovial Sarcoma; T-lymphoma; T-Cell Childhood Acute Lymphoblastic Leukemia; Testicular Cancer; Thorax and Respiratory Cancer; Throat Cancer; Thyroid Cancer; Transitional Cell Cancer of the Renal Pelvis and Ureter; Transitional Cell Carcinoma of the Bladder; Tubal Carcinoma; Unresectable or Metastatic Malignant Gastrointestinal Stromal Tumor (GIST); Unspecified Childhood Solid Tumor; Unspecified Adult Solid Tumor; Untreated Childhood Brain Stem Glioma; Urethral Cancer; Uterine Carcinosarcoma; Uterine Sarcoma; and Wilm's Tumor.

### VIII. METHODS OF ADMINISTRATION

As explained above, the present invention is directed towards methods for modulating tumor growth and metastasis comprising, inter alia, the administration of a VDA and a RTK inhibitor. The agents of the invention can be administered separately (e.g, formulated and administered separately), or in combination as a pharmaceutical composition of the present invention. Administration can be achieved by any suitable route, such as parenterally, transmucosally, e.g., orally, nasally, or rectally, or transdermally. Preferably, administration is parenteral, e.g., via intravenous injection. Alternative means of administration also include, but are not limited to, intra-arteriole, intramuscular, intradermal, subcutaneous, intraperitoneal, intraventricular, and intracranial administration/or by injection into the tumor(s) being treated or into tissues surrounding the tumor(s).

The pharmaceutical composition may be employed in any suitable pharmaceutical formulation, as described above, including in a vesicle, such as a liposome [see Langer, Science 249:1527-1533 (1990); Treat et al.ᵣ in Liposomes in the Therapy of Infectious Disease and Cancer, Lopez-Berestein and Fidler (eds.), Liss: New York, pp. 317-327, see generally, ibid] Preferably, administration of liposomes containing the agents of the invention is parenteral, e.g., via intravenous injection, but also may include, without limitation, intra-arteriole, intramuscular, intradermal, subcutaneous, intraperitoneal, intraventricular, and intracranial administration, or by injection into the tumor(s) being treated or into tissues surrounding the tumor(s).

In yet another embodiment, a pharmaceutical composition of the present invention can be delivered in a controlled release system, such as using an intravenous infusion, an implantable osmotic pump, a transdermal patch, liposomes, or other modes of administration. In a particular embodiment, a pump may be used [see Langer, supra; Sefton, CRC Crit. Ref. Biomed. Eng. 14:201 (1987); Buchwald et al., Surgery_{—}88:507 (1980); Saudek et al., N. Engl. J. Med. 321:574 (1989)]. In another embodiment, polymeric materials can be used [see Medical Applications of Controlled Release, Langer and Wise (eds.)/CRC Press: Boca Raton, Fla. (1974); Controlled Drug Bioavailability, Drug Product Design and Performance, Smolen and Ball (eds.), Wiley: New York (1984); Ranger and Peppas, J. Macromol. Sci. Rev. Macromol. Chem. 23:61 (1983); see also Levy et al., Science 228:190 (1985); During et al., Ann. Neurol. 25:351 (1989); Howard et al., J. Neurosurg. 71:105 (1989)]. In yet another embodiment, a controlled release system can be placed in proximity of the target tissues of the animal, thus requiring only a fraction of the systemic dose [see, e.g., Goodson, in Medical Applications of Controlled Release, supra, vol. 2, pp. 115-138 (1984).]. In particular, a controlled release device can be introduced into an animal in proximity of the site of inappropriate immune activation or a tumor. Other controlled release systems are discussed in the review by Langer [Science 249:1527-1533 (1990)].

A controlled release formulation can be pulsed, delayed, extended, slow, steady, immediate, rapid, fast, etc. It can comprise one or more release formulations, e.g. extended- and immediate-release components. Extended delivery systems can be utilized to achieve a dosing internal of once every 24 hours, once every 12 hours, once every 8 hours, once every 6 hours, etc. The dosage form/delivery system can be a tablet or a capsule suited for extended release, but a sustained release liquid or suspension can also be used. A controlled release pharmaceutical formulation can be produced which maintains the release of, and or peak blood plasma levels of a compound of the invention.

Compounds of the invention may also be administrated transdermally using methods known to those skilled in the art (see, for example: Chien; “Transdermal Controlled Systemic Medications”; Marcel Dekker, Inc.; 1987. Lipp et al. WO94/04157 3 Mar. 1994). For example, a solution or suspension of a compound of the invention in a suitable volatile solvent optionally containing penetration enhancing agents can be combined with additional additives known to those skilled in the art, such as matrix materials and bacteriocides. After sterilization, the resulting mixture can be formulated following known procedures into dosage forms. In addition, on treatment with emulsifying agents and water, a solution or suspension of a compound of the invention may be formulated into a lotion or salve.

Suitable solvents for processing transdermal delivery systems are known to those skilled in the art, and include lower alcohols such as ethanol or isopropyl alcohol, lower ketones such as acetone, lower carboxylic acid esters such as ethyl acetate, polar ethers such as tetrahydrofuran, lower hydrocarbons such as hexane, cyclohexane or benzene, or halogenated hydrocarbons such as dichloromethane, chloroform, trichlorotrifluoroethane, or trichlorofluoroethane. Suitable solvents may also include mixtures of one or more materials selected from lower alcohols, lower ketones, lower carboxylic acid esters, polar ethers, lower hydrocarbons, halogenated hydrocarbons.

Suitable penetration enhancing materials for transdermal delivery system are known to those skilled in the art, and include, for example, monohydroxy or polyhydroxy alcohols such as ethanol, propylene glycol or benzyl alcohol, saturated or unsaturated C8-C 18 fatty alcohols such as lauryl alcohol or cetyl alcohol, saturated or unsaturated C8-C 18 fatty acids such as stearic acid, saturated or unsaturated fatty esters with up to 24 carbons such as methyl, ethyl, propyl, isopropyl, n-butyl, sec-butyl, isobutyl, tertbutyl or monoglycerin esters of acetic acid, capronic acid, lauric acid, myristinic acid, stearic acid, or palmitic acid, or diesters of saturated or unsaturated dicarboxylic acids with a total of up to 24 carbons such as diisopropyl adipate, diisobutyl adipate, diisopropyl sebacate, diisopropyl maleate, or diisopropyl fumarate. Additional penetration enhancing materials include phosphatidyl derivatives such as lecithin or cephalin, terpenes, amides, ketones, ureas and their derivatives, and ethers such as dimethyl isosorbid and diethyleneglycol monoethyl ether. Suitable penetration enhancing formulations may also include mixtures of one or more materials selected from monohydroxy or polyhydroxy alcohols, saturated or unsaturated C 8-C18 fatty alcohols, saturated or unsaturated 08-C 18 fatty acids, saturated or unsaturated fatty esters with up to 24 carbons, diesters of saturated or unsaturated discarboxylic acids with a total of up to 24 carbons, phosphatidyl derivatives, terpenes, amides, ketones, ureas and their derivatives, and ethers.

Suitable binding materials for transdermal delivery systems are known to those skilled in the art and include polyacrylates, silicones, polyurethanes, block polymers, styrenebutadiene copolymers, and natural and synthetic rubbers. Cellulose ethers, derivatized polyethylenes, and silicates may also be used as matrix components. Additional additives, such as viscous resins or oils may be added to increase the viscosity of the matrix.

### IX. SYNTHETIC PROCEDURE

Compounds of the present invention are prepared from commonly available compounds using procedures known to those skilled in the art, including any one or more of the following conditions without limitation:

Within the scope of this text, only a readily removable group that is not a constituent of the particular desired end product of the compounds of the present invention is designated a “protecting group,” unless the context indicates otherwise. The protection of functional groups by such protecting groups, the protecting groups themselves, and their cleavage reactions are described for example in standard reference works, such as e.g., Science of Synthesis: Houben-Weyl Methods of Molecular Transformation. Georg Thieme Verlag, Stuttgart, Germany. 2005. 41627 pp. (URL: http://www.science-of-synthesis.com (Electronic Version, 48 Volumes)); J. F. W. McOmie, “Protective Groups in Organic Chemistry”, Plenum Press, London and New York 1973, in T. W. Greene and P. G. M. Wuts, “Protective Groups in Organic Synthesis”, Third edition, Wiley, New York 1999, in “The Peptides”; Volume 3 (editors: E. Gross and J. Meienhofer), Academic Press, London and New York 1981, in “Methoden der organischen Chemie” (Methods of Organic Chemistry), Houben Weyl, 4th edition, Volume 15/I, Georg Thieme Verlag, Stuttgart 1974, in H.-D. Jakubke and H. Jeschkeit, “Aminosäuren, Peptide, Proteine” (Amino acids, Peptides, Proteins), Verlag Chemie, Weinheim, Deerfield Beach, and Basel 1982, and in Jochen Lehmann, “Chemie der Kohlenhydrate: Monosaccharide und Derivate” (Chemistry of Carbohydrates: Monosaccharides and Derivatives), Georg Thieme Verlag, Stuttgart 1974. A characteristic of protecting groups is that they can be removed readily (i.e., without the occurrence of undesired secondary reactions) for example by solvolysis, reduction, photolysis or alternatively under physiological conditions (e.g., by enzymatic cleavage).

Acid addition salts of the compounds of the invention are most suitably formed from pharmaceutically acceptable acids, and include for example those formed with inorganic acids e.g. hydrochloric, hydrobromic, sulphuric or phosphoric acids and organic acids e.g. succinic, malaeic, acetic or fumaric acid. Other non-pharmaceutically acceptable salts e.g. oxalates can be used for example in the isolation of the compounds of the invention, for laboratory use, or for subsequent conversion to a pharmaceutically acceptable acid addition salt. Also included within the scope of the invention are solvates and hydrates of the invention.

The conversion of a given compound salt to a desired compound salt is achieved by applying standard techniques, in which an aqueous solution of the given salt is treated with a solution of base e.g. sodium carbonate or potassium hydroxide, to liberate the free base which is then extracted into an appropriate solvent, such as ether. The free base is then separated from the aqueous portion, dried, and treated with the requisite acid to give the desired salt.

In vivo hydrolyzable esters or amides of certain compounds of the invention can be formed by treating those compounds having a free hydroxy or amino functionality with the acid chloride of the desired ester in the presence of a base in an inert solvent such as methylene chloride or chloroform. Suitable bases include triethylamine or pyridine. Conversely, compounds of the invention having a free carboxy group can be esterified using standard conditions which can include activation followed by treatment with the desired alcohol in the presence of a suitable base.

Examples of pharmaceutically acceptable addition salts include, without limitation, the non-toxic inorganic and organic acid addition salts such as the hydrochloride derived from hydrochloric acid, the hydrobromide derived from hydrobromic acid, the nitrate derived from nitric acid, the perchlorate derived from perchloric acid, the phosphate derived from phosphoric acid, the sulphate derived from sulphuric acid, the formate derived from formic acid, the acetate derived from acetic acid, the aconate derived from aconitic acid, the ascorbate derived from ascorbic acid, the benzenesulphonate derived from benzenesulphonic acid, the benzoate derived from benzoic acid, the cinnamate derived from cinnamic acid, the citrate derived from citric acid, the embonate derived from embonic acid, the enantate derived from enanthic acid, the fumarate derived from fumaric acid, the glutamate derived from glutamic acid, the glycolate derived from glycolic acid, the lactate derived from lactic acid, the maleate derived from maleic acid, the malonate derived from malonic acid, the mandelate derived from mandelic acid, the methanesulphonate derived from methane sulphonic acid, the naphthalene-2-sulphonate derived from naphtalene-2-sulphonic acid, the phthalate derived from phthalic acid, the salicylate derived from salicylic acid, the sorbate derived from sorbic acid, the stearate derived from stearic acid, the succinate derived from succinic acid, the tartrate derived from tartaric acid, the toluene-p-sulphonate derived from p-toluene sulphonic acid, and the like. Particularly preferred salts are sodium, lysine and arginine salts of the compounds of the invention. Such salts can be formed by procedures well known and described in the art.

Other acids such as oxalic acid, which can not be considered pharmaceutically acceptable, can be useful in the preparation of salts useful as intermediates in obtaining a chemical compound of the invention and its pharmaceutically acceptable acid addition salt.

Metal salts of a chemical compound of the invention include alkali metal salts, such as the sodium salt of a chemical compound of the invention containing a carboxy group.

Mixtures of isomers obtainable according to the invention can be separated in a manner known per se into the individual isomers; diastereoisomers can be separated, for example, by partitioning between polyphasic solvent mixtures, recrystallisation and/or chromatographic separation, for example over silica gel or by, e.g., medium pressure liquid chromatography over a reversed phase column, and racemates can be separated, for example, by the formation of salts with optically pure salt-forming reagents and separation of the mixture of diastereoisomers so obtainable, for example by means of fractional crystallisation, or by chromatography over optically active column materials.

Intermediates and final products can be worked up and/or purified according to standard methods, e.g., using chromatographic methods, distribution methods, (re-) crystallization, and the like.

The following examples are provided to illustrate embodiments of the invention. They are not intended to limit the invention in any way.

### EXAMPLES

### Example 1

### Enhancement of In Vivo Anti-Tumor Effect in a Mouse Model of Melanoma Following VDA+RTK Inhibitor Combination Therapy

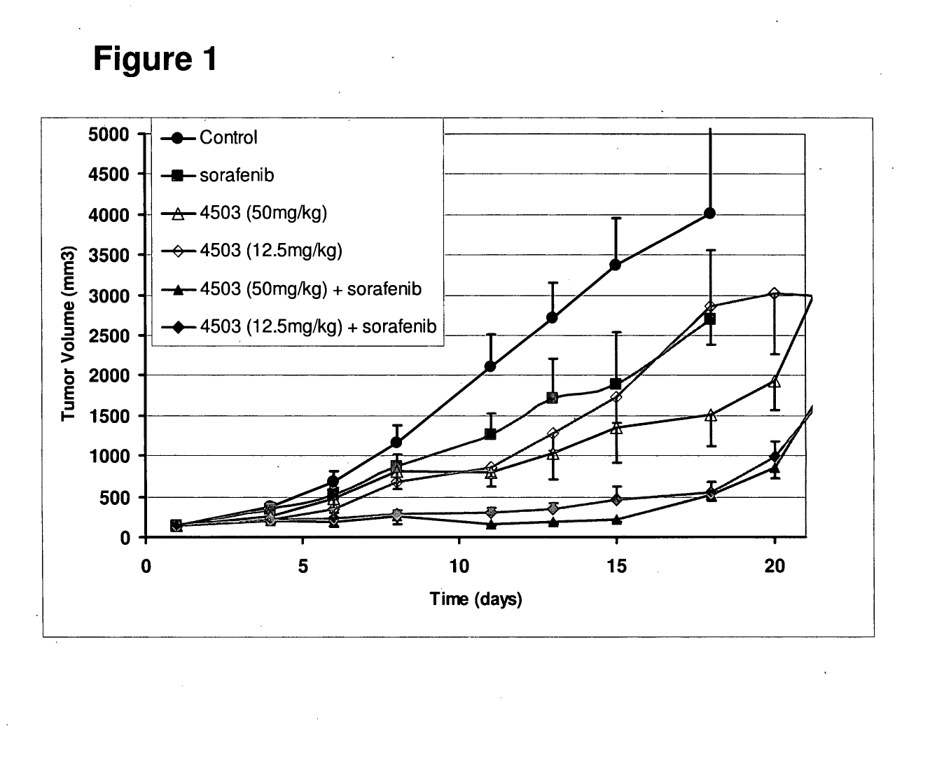

CA1P (OXiGENE, Waltham, Mass.) was administered alone and in combination with Sorafenib (Nexavarm™, Bayer Pharmaceuticals, Germany) for potential antitumor activity against LOX IMVI human melanoma xenografts in athymic nude mice (NCr-nu/nu). There were six treatment groups of 10 mice each. There was one-vehicle-treated control group (“control”). In a second treatment group, Sorafenib was delivered daily for 14 days (q1d×14) by oral gavage (p.o.) at a dose of 40 mg/kg (“Sorafenib”). In the third and fourth treatment groups, CA1P was delivered once a week for two weeks (q7d×2) by intraperitoneal (i.p.) injection at two different doses (“4503 50 mg/kg” and “4503 12.5 mg/kg”, respectively). In the fifth and sixth treatment groups, each dosage of CA1P was also combined with Sorafenib (“4503 50 mg/kg+Sorafenib” and “4503 12.5 mg/kg+Sorafenib”). Both of the combination treatment groups exhibited enhanced antitumor activity relative to the single agent treatment groups (see FIG. 1).

### Example 2

### Synergistic Enhancement of In Vivo Anti-Tumor Effect in a Mouse Model of Liver Cancer Following VDA+RTK Inhibitor Combination Therapy

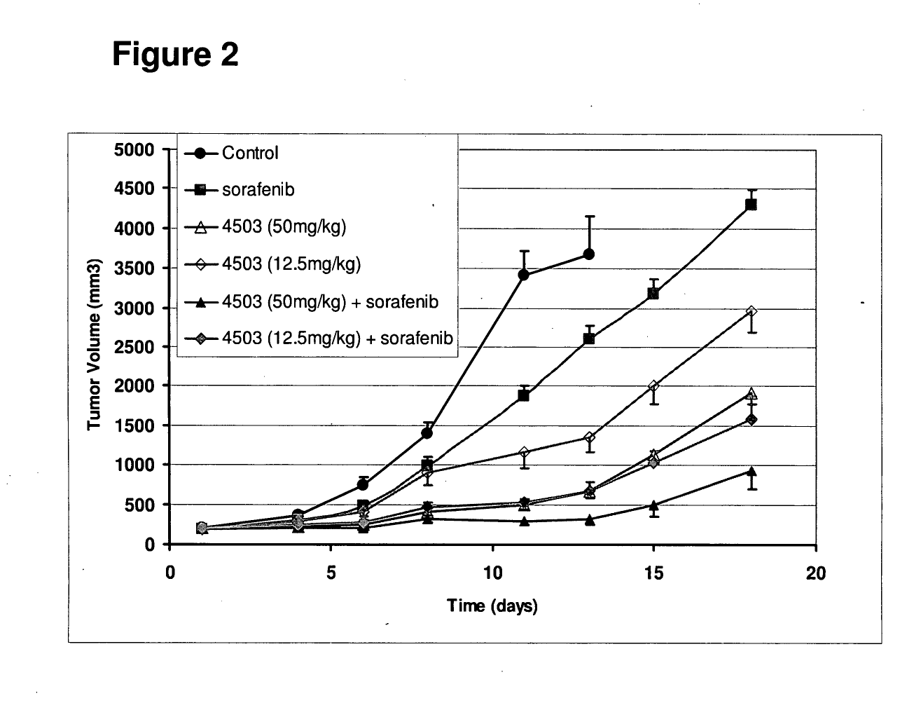

CA1P was administered alone and in combination with Sorafenib for potential antitumor activity against HEP-G2 human hepatocellular carcinoma xenografts in Scid mice. There were six treatment groups of 10 mice each. There was one-vehicle-treated control group (“control”). In a second treatment group, Sorafenib was delivered daily for 14 days (q1d×14) by oral gavage (p.o.) at a dose of 40 mg/kg (“Sorafenib”). In the third and fourth treatment groups, CA1P was delivered once a week for two weeks (q7d×2) by intraperitoneal (i.p.) injection at two different doses (“4503 50 mg/kg” and “4503 12.5 mg/kg”, respectively). In the fifth and sixth treatment groups, each dosage of CA1P was also combined with sorafenib (“4503 50 mg/kg+Sorafenib” and “4503 12.5 mg/kg+Sorafenib”). Both of the combination treatment groups exhibited enhanced antitumor activity relative to the single agent treatment groups (see FIG. 2).

### Example 3

### Synergistic Enhancement of In Vivo Anti-Tumor Effect in a Mouse Model of Lung Cancer Following VDA+RTK Inhibitor Combination Therapy

CA1P and CA4P are administered alone and in combination with Erlotinib (Tarceva™, OSI Pharmaceuticals) for potential antitumor activity against H460 lung xenografts in Scid mice. There are 6 treatment groups of 10 mice each. There is one-vehicle-treated control group (“control”). In a second treatment group, CA4P is administered at 75 mg/kg (i.p.) once a week for 3 weeks. In a third treatment group, CA1P is administered at 75 mg/kg (i.p.) once a week for 3 weeks. In a fourth treatment group, Erlotinib is delivered daily for 3 weeks by oral gavage (p.o.) at a dose of 50 mg/kg (“Erlotinib”). In the fifth treatment group, CA4P is combined with Erlotinib at the same dosages used in treatment groups two and four. In the sixth treatment group, CA1P is combined with Erlotinib at the same dosages used in treatment groups three and four.

Those skilled in the art will recognize, or be able to ascertain using no more than routine experimentation, many equivalents to the specific embodiments of the invention described herein. Such equivalents are intended to be encompassed by the following claims.

All publications and patent documents cited herein are hereby incorporated by reference in their entirety for all purposes to the same extent as if each were so individually denoted.

What is claimed is:

1. A method for producing an anti-tumor effect in a subject suffering from cancer the method comprising administering to the subject a Vascular Disrupting Agent (VDA) and a small molecule Receptor Tyrosine Kinase (RTK) Inhibitor in amounts effective therefor.

2. A method for inhibiting tumor-associated angiogenesis in a subject treated with a VDA, the method comprising administering to the subject a small molecule RTK Inhibitor in amounts effective therefor.

3. A method for preventing tumor regrowth in a subject suffering from cancer, the method comprising administering to the subject a Vascular Disrupting Agent (VDA) and a small molecule RTK inhibitor in amounts effective therefor.

4. The method of any one of claim 1, wherein the RTK inhibitor is an inhibitor of at least one receptor tyrosine kinase (RTK) selected from the group consisting of: an RTK of the VEGF receptor family, an RTK of the EGF receptor family, an RTK of the RET receptor family, and an RTK of the PDGF receptor family.

5. The method of any one of claim 1, wherein the RTK inhibitor is selected from the group consisting of Axitinib, Cediranib, Dasatinib, Erlotinib, Gefitinib, Imatinib, Lapatinib, Lestaurtinib, Nilotinib, Pazopanib, Semaxinib, Sorafenib, Sunitinib, Vatalanib, and Vandetanib.

6. The method of claim 5, wherein the RTK inhibitor is Sorafenib.

7. The method of claim 5, wherein the RTK inhibitor is Erlotinib.

8. The method of claim 1, wherein the VDA is a combretastatin agent.

9. The method of claim 8, wherein the combretastatin agent is Combretastatin A1 (CA1) or a prodrug or pharmaceutically acceptable salt thereof.

10. The method of claim 8, wherein the combretastatin agent is Combretastatin A4 (CA4) or a prodrug or pharmaceutically acceptable salt thereof.

11. The method of claim 8, wherein the combretastatin agent is a phosphate prodrug.

12. The method of claim 11, wherein the combrestatin agent is a compound of Formula II: 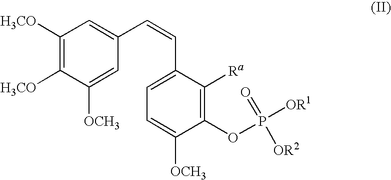
wherein R^{a} is H or OP(O)(OR³)OR⁴; and
OR¹, OR², OR³ and OR⁴ are each, independently, H, —O⁻QH⁺ or —O⁻M⁺,
wherein M⁺ is a monovalent or divalent metal cation, and Q is, independently:
a) an amino acid containing at least two nitrogen atoms where one of the nitrogen atoms, together with a proton, forms a quaternary ammonium cation QH⁺; or
b) an organic amine containing at least one nitrogen atom which, together with a proton, forms a quaternary ammonium cation, QH⁺.

13. The method of claim 12, wherein Formula II is represented by a compound of Formula III: 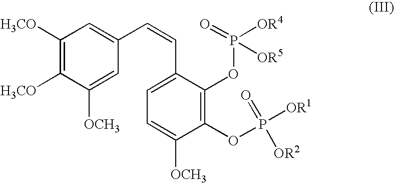
wherein OR¹, OR², OR³ and OR⁴ are each, independently, H, —O⁻QH⁺ or —O⁻M⁺,
wherein M⁺ is a monovalent or divalent metal cation, and Q is, independently:
a) an amino acid containing at least two nitrogen atoms where one of the nitrogen atoms, together with a proton, forms a quaternary ammonium cation QH⁺; or
b) an organic amine containing at least one nitrogen atom which, together with a proton, forms a quaternary ammonium cation, QH⁺.

14. The method of claim 12, wherein the metal cation is sodium or potassium.

15. The method of claim 12, wherein the organic amine is TRIS.

16. The method of claim 1, wherein the VDA and RTK inhibitor are simultaneously or sequentially administered.

17. The method of claim 1, wherein said cancer is selected from the group consisting of ovarian cancer, fallopian tube cancer, cervical cancer, breast cancer, liver cancer, small cell lung cancer, non-small cell lung cancer, skin cancer, colorectal cancer, esophageal cancer, gastric cancer, leukemia, renal cancer, head and neck cancer, glioma, pancreatic cancer, lymphoma, prostate cancer, and primary cancer of the peritoneum.

18. The method of claim 17, wherein said cancer is skin cancer or liver cancer.

19. A method of treating a subject suffering from cancer, the method comprising administering to the subject a small molecule RTK inhibitor and a combretastatin agent in amounts effective therefor.

20. The method of claim 19, wherein the RTK inhibitor and the combretastatin agent are administered simultaneously.

21. The method of claim 19, wherein the RTK inhibitor is Sorafenib.

22. The method of claim 19, wherein the RTK inhibitor is Erlotinib.

23. The method of claim 19, wherein the combretastatin agent is represented by Formula II.

24. A method of treating a subject suffering from cancer, the method comprising thereof by administering to the subject a pharmaceutical composition comprising effective amounts of Sorafenib and CA1P.

25. A method of treating a subject suffering from cancer, the method comprising thereof by administering to the subject a pharmaceutical composition comprising effective amounts of Sorafenib and CA4P.

26. A method of treating a subject suffering from cancer, the method comprising thereof by administering to the subject a pharmaceutical composition comprising effective amounts of Erlotinib and CA1P.

27. A method of treating a subject suffering from cancer, the method comprising thereof by administering to the subject a pharmaceutical composition comprising effective amounts of Erlotinib and CA4P.

28. A pharmaceutical composition for producing an anti-tumor effect in a subject suffering from cancer, comprising effective amounts of a Vascular Disrupting Agent (VDA) and a small molecule RTK inhibitor in a pharmaceutical carrier.

29. The composition of claim 28, wherein the RTK inhibitor is an inhibitor of at least one receptor tyrosine kinase (RTK) selected from the group consisting of: an RTK of the VEGF receptor family, an RTK of the EGF receptor family, an RTK of the RET receptor family, and an RTK of the PDGF receptor family.

30. The composition of claim 29, wherein the RTK inhibitor is selected from the group consisting of Axitinib, Cediranib, Dasatinib, Erlotinib, Gefitinib, Imatinib, Lapatinib, Lestaurtinib, Nilotinib, Pazopanib, Semaxinib, Sorafenib, Sunitinib, Vatalanib and Vandetanib.

31. The composition of claim 30, wherein the RTK inhibitor is Sorafenib.

32. The composition of claim 30, wherein the RTK inhibitor is Erlotinib.

33. The composition of claim 28, wherein the VDA is a combretastatin agent.

34. The composition of claim 28, wherein the combretastatin agent is Combretastatin A1 (CA1) or a prodrug or pharmaceutically acceptable salt thereof.

35. The composition of claim 28, wherein the combretastatin agent is Combretastatin A4 (CA4) or a prodrug or pharmaceutically acceptable salt thereof.

36. The composition of claim 28, wherein the combretastatin agent is a phosphate prodrug.

37. The composition of claim 36, wherein the combretastatin agent is a compound of Formula II: 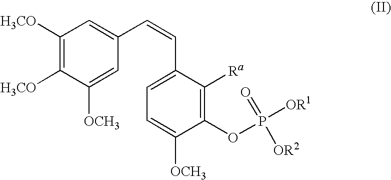
wherein R^{a} is H or OP(O)(OR³)OR⁴; and
OR¹, OR², OR³ and OR⁴ are each, independently, H, —O⁻QH⁺ or —O⁻M⁺,
wherein M⁺ is a monovalent or divalent metal cation, and Q is, independently:
a) an amino acid containing at least two nitrogen atoms where one of the nitrogen atoms, together with a proton, forms a quaternary ammonium cation QH⁺; or
b) an organic amine containing at least one nitrogen atom which, together with a proton, forms a quaternary ammonium cation, QH⁺.

38. The composition of claim 37, wherein Formula II is represented by a compound of Formula III: 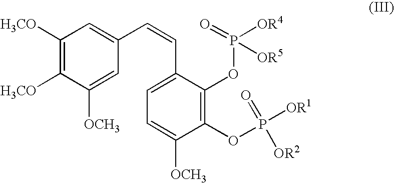
wherein OR¹, OR², OR³ and OR⁴ are each, independently, H, —O⁻QH⁺ or —O⁻M⁺,
wherein M⁺ is a monovalent or divalent metal cation, and Q is, independently:
a) an amino acid containing at least two nitrogen atoms where one of the nitrogen atoms, together with a proton, forms a quaternary ammonium cation QH⁺; or
b) an organic amine containing at least one nitrogen atom which, together with a proton, forms a quaternary ammonium cation, QH⁺.

39. The composition of claim 37, wherein the metal cation is sodium or potassium.

40. The composition of claim 37, wherein the organic amine is TRIS.

41. A pharmaceutical composition for producing an anti-tumor effect in a subject suffering from cancer, comprising effective amounts of Sorafenib and CA1P in a pharmaceutical carrier.

42. A pharmaceutical composition for producing an anti-tumor effect in a subject suffering from cancer, comprising effective amounts of Erlotinib and CA1P in a pharmaceutical carrier.

43. A pharmaceutical composition for producing an anti-tumor effect in a subject suffering from cancer, comprising effective amounts of Sorafenib and CA4P in a pharmaceutical carrier.

44. A pharmaceutical composition for producing an anti-tumor effect in a subject suffering from cancer, comprising effective amounts of Erlotinib and CA4P in a pharmaceutical carrier.
